(19) United States
(12) Patent Application Publication
(10) Pub. No.: US 2003/0228617 A1
(43) Pub. Date: Dec. 11, 2003

(54) Method for predicting autoimmune diseases

(75) Inventors: Thomas M. Aune, Franklin, TN (US); Nancy J. Olsen, Nashville, TN (US)

Correspondence Address: JENKINS & WILSON, PA, 3100 TOWER BLVD, SUITE 1400, DURHAM, NC, 27707, US

(73) Assignee: Vanderbilt University

(21) Appl. No.: 10/439,388

(22) Filed: May 16, 2003

Related U.S. Application Data
(60) Provisional application No. 60/381,055, filed on May 16, 2002.

(51) Int. Cl. C12Q 1/68; C12P 19/34

(52) U.S. Cl. 435/6; 435/91.2

(57) ABSTRACT

The presently claimed subject matter provides a method for detecting an autoimmune disorder in a subject by obtaining a biological sample from the subject; determining expression levels of at least two genes in the biological sample; and comparing the expression level of each gene with a standard, wherein the comparing detects the presence of an autoimmune disorder in the subject. Also provided are compositions and kits for carrying out the methods of the presently claimed subject matter.

### CROSS REFERENCE TO RELATED APPLICATIONS

This application is based on and claims priority to U.S. Provisional Application Serial No. 60/381,055, filed May 16, 2002, herein incorporated by reference in its entirety.

### GRANT STATEMENT

This work was supported by grants A144924, AR02027, AR41943, and DK58765 from the U.S. National Institutes of Health. Thus, the U.S. government has certain rights in the presently claimed subject matter.

### TECHNICAL FIELD

The presently claimed subject matter generally relates to the diagnosis of autoimmune disease. More specifically, this presently claimed subject matter relates to identifying a reduced probability of having an autoimmune disease, such as systemic lupus erythematosus, rheumatoid arthritis, multiple sclerosis, or Type 1 diabetes.

Table of Abbreviations

| | | |
|---|---|---|
| 6-JOE | - | 6-carboxy-4′,5′-dichloro-2′,7′- |
| | | dimethoxyfluorescein, succinimidyl ester |
| aaRNA | - | amplified antisense RNA |
| Ags | - | antigens |
| AP3S2 | - | adaptor-related protein complex 3, sigma 2 |
| | | subunit |
| ASL | - | argininosuccinate lyase |
| BMP8 | - | bone morphogenetic protein 8 (osteogenic |
| | | protein 2) |
| BPHL | - | biphenyl hydrolase-like (serine hydrolase; breast |
| | | epithelial mucin-associated antigen) |
| BRCA1 | - | breast cancer 1, early onset, transcript variant |
| | | BRCA1a |
| CASP6 | - | caspase 6 |
| CDH1 | - | cadherin 1, type 1, E-cadherin (epithelial) |
| CDKN1B | - | cyclin-dependent kinase inhibitor 1B |
| cDNA | - | complementary DNA |
| CYB5-M | - | cytochrome b5 outer mitochondrial membrane |
| | | precursor |
| DEPC | - | diethylpyrocarbonate |
| DIPA | - | hepatitis delta antigen-interacting protein A |
| DMARDs | - | disease-modifying anti-rheumatic drugs |
| DNAJA1 | - | DnaJ homolog, subfamily A, member 1 |
| EPB72 | - | erythrocyte membrane protein band 7.2 |
| | | (stomatin) |
| EST | - | expressed sequence tag |
| FITC | - | fluorescein isothiocyanate |
| GMBS | - | gamma-maleimidobutyryloxy-succimide |
| GNB5 | - | human guanine nucleotide binding protein, beta 5 |
| GUCY1B3 | - | guanylate cyclase 1, soluble, beta 3 |
| HSJ2 | - | heat shock protein, DNAJ-like 2 |
| IDDM | - | insulin-dependent (type 1) diabetes mellitus |
| IFN | - | interferon |
| LabMAP | - | Laboratory Multiple Analyte Profiling |
| LIF | - | leukemia inhibitory factor |
| LLGL2 | - | lethal giant larvae homolog 2 |
| MAN1A1 | - | mannosidase, alpha, class 1A, member 1 |
| MMP17 | - | matrix metalloproteinase 17 |
| MS | - | multiple sclerosis |
| MYO1C | - | myosin I C |
| NSAIDs | - | nonsteroidal anti-inflammatory drugs |
| ORC1L | - | origin recognition complex, subunit 1-like |
| PCR | - | polymerase chain reaction |
| PMBC | - | peripheral blood mononuclear cell(s) |
| RA | - | rheumatoid arthritis |
| RAPD | - | rapid amplification of polymorphic DNA |
| ROCK | - | Random Oligonucleotide Construction Kit |
| RTN4 | - | reticulon 4 |
| RT-PCR | - | reverse transcription PCR |
| SC65 | - | synaptonemal complex protein 65 |
| SD | - | standard deviation(s) |
| SIP1 | - | survival of motor neuron protein interacting |
| | | protein 1 |
| SISPA | - | Sequence-Independent, Single-Primer |
| | | Amplification |
| SLC16A4 | - | solute carrier family 16, member 4 |
| SLE | - | systemic lupus erythematosus |
| SSP29 | - | silver-stainable protein 29, also called acidic |
| | | (leucine-rich) nuclear phosphoprotein 32 |
| | | family, member B |
| STOM | - | alternate abbreviation for stomatin |
| SUDD | - | human sudD suppressor of bimD6 homolog |
| | | (SUDD) from Aspergillus nidulans, transcript |
| | | variant 1 |
| TAF11 | - | TATA box binding protein- associated factor 11 |
| TAF2I | - | TAF11 RNA polymerase II, TATA box binding |
| | | protein-associated factor, 28 kilodalton |
| TBP | - | TATA box binding protein |
| TGM2 | - | transglutaminase 2 |
| TNF-α | - | tumor necrosis factor alpha |
| TNFAIP2 | - | tumor necrosis factor, alpha-induced protein 2 |
| TP53 | - | human tumor protein p53 (Li-Fraumeni |
| | | syndrome) |
| TXK | - | TXK tyrosine kinase |
| UBE2G2 | - | ubiquitin-conjugating enzyme E2G 2 (UBC7 |
| | | homolog, yeast) |

Amino Acid Abbreviations and Corresponding mRNA Codons

| Amino Acid | 3-Letter | 1-Letter | mRNA Codons |
|---|---|---|---|
| Alanine | Ala | A | GCA GCC GCG GCU |
| Arginine | Arg | R | AGA AGG CGA CGC CGG CGU |
| Asparagine | Asn | N | AAC AAU |
| Aspartic Acid | Asp | D | GAC GAU |
| Cysteine | Cys | C | UGC UGU |
| Glutamic Acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG CUU |
| Lysine | Lys | K | AAA AAG |
| Methionine | Met | M | AUG |
| Proline | Pro | P | CCA CCC CCG CCU |
| Phenylalanine | Phe | F | UUC UUU |
| Serine | Ser | S | ACG AGU UCA UCC UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

### BACKGROUND ART

Autoimmune diseases affect millions of people in the United States, with approximately 3-5% of the population being affected. See Jacobson et al., 1997; Marrack et al., 2001. The pathogenesis of autoimmune disease generally involves an attack by the patient's immune system on an organ or tissue, such as seen in cases of type 1 (insulin-dependent) diabetes (pancreatic β cells; see Kukreja & Maclaren 2000), multiple sclerosis (myelin basic protein; see Ufret-Vincenty et al., 1998), and thyroiditis (thyroglobulin or thyroid peroxidase; see Martin et al., 1999). Certain autoimmune diseases are also characterized by systemic attacks, including immunological responses against the synovial lining, lung, and heart in rheumatoid arthritis (see Quayle et al., 1992) and the skin, kidney, and heart in systemic lupus erythematosus (see Kotzin 1996).

Classification of disease syndromes, prediction of disease course, and understanding disease pathogenesis are three fundamental goals of research in autoimmunity. Diagnosis of autoimmune diseases often requires several patient visits to the doctor and repeated clinical testing. This is largely due to the fact that no single test or combination of clinical tests presently available is an absolute predictor of autoimmune disease. For example, reliably establishing a diagnosis of rheumatoid arthritis (RA) using existing criteria requires a history of at least 3 months of symptoms.

The importance of the need for a rapid and accurate diagnostic test for autoimmune diseases is underscored by changes in the approaches to treatment of these diseases. Until recently, rheumatologists initiated therapy for a newly diagnosed patient with nonsteroidal anti-inflammatory drugs (NSAIDs) and low dose corticosteroids. As the disease progressed, additional disease modifying anti-rheumatic drugs (DMARDs) were added. Rheumatologists now recognize that early and aggressive therapy with newer agents such as methotrexate, leflunomide, or the new tumor necrosis factor-α (TNF-α) inhibitors (for example, etanercept and infliximab) can provide improved outcomes and actually preserve function and improve quality of life. See Jacobson et al., 1997. However, these newer drugs are expensive and can result in significant side effects, and thus are better used in patients that clearly have RA.

Therefore, improved diagnostic tests that can readily exclude an individual from the classification of having an autoimmune disease are needed. This and other needs in the art are addressed by the present disclosure.

### SUMMARY

The presently claimed subject matter provides method and compositions for detecting an autoimmune disorder in a subject. In one embodiment, the method comprises (a) obtaining a biological sample from the subject; (b) determining expression levels of at least two genes in the biological sample; and (c) comparing the expression level of each gene determined in step (b) with a standard, wherein the comparing detects the presence of an autoimmune disorder in the subject. In one embodiment, the autoimmune disorder is selected from the group consisting of rheumatoid arthritis (RA), systemic lupus erythematosus (SLE), multiple sclerosis (MS), type 1 (i.e. insulin-dependent) diabetes (IDDM), and combinations thereof. In one embodiment, the biological sample is a cell. In one embodiment, the cell is a peripheral blood mononuclear cell. In one embodiment, the subject is an animal. In one embodiment, the animal is a mammal. In one embodiment, the mammal is a human. In one embodiment of the present method, the determining in step (b) comprises a technique selected from the group consisting of a Northern blot, hybridization to a nucleic acid microarray, and a reverse transcription-polymerase chain reaction (RT-PCR). In one embodiment, the RT-PCR is quantitative RT-PCR.

In alternative embodiments of the present method, the determining in step (b) is of the expression levels of at least two genes, of at least five genes, of at least ten genes, of at least twenty genes, of at least twenty-five genes, or of all of the genes identified in SEQ ID NOs: 1-70.

In accordance with the methods of the presently claimed subject matter, in one embodiment the comparing comprises: (a) establishing an average expression level for each gene in a population, wherein the population comprises statistically significant numbers of normal subjects and subjects that have one or more different autoimmune disorders; (b) assigning a first value to each gene for which the expression level in the subject is higher than the average expression level in the population and a second value to each gene for which the expression level in the subject is lower than the average expression level in the population; and (c) adding the values assigned in step (b) to arrive at a sum, wherein the sum is indicative of the presence or absence of an autoimmune disorder in the subject.

The presently claimed subject matter also provides a method of diagnosing an autoimmune disorder in a subject comprising: (a) providing an array comprising a plurality of nucleic acid sequences, wherein each nucleic acid sequence corresponds to a known gene; (b) providing a biological sample derived from the subject, wherein the biological sample comprises a nucleic acid; (c) hybridizing the biological sample to the array; (d) detecting all nucleic acids on the array to which the biological sample hybridizes; (e) determining a relative expression level for each nucleic acid detected; (f) creating a profile of the relative expression levels for the detected nucleic acids; and (g) comparing the profile created with a standard profile, wherein the comparing diagnoses an autoimmune disease in a subject. In one embodiment, the autoimmune disorder is selected from the group consisting of rheumatoid arthritis (RA), systemic lupus erythematosus (SLE), multiple sclerosis (MS), type 1 (insulin-dependent) diabetes (IDDM), and combinations thereof. In one embodiment, the array is selected from the group consisting of a microarray chip and a membrane-based filter array. In alternative embodiments, the array comprises at least two genes, at least five genes, at least ten genes, at least twenty genes, at least twenty-five genes, or all of the genes identified in SEQ ID NOs: 1-70. In another embodiment, the array further comprises at least one internal control gene. In one embodiment, the biological sample is a cell. In one embodiment, the cell is a peripheral blood mononuclear cell. In one embodiment, the subject is an animal. In one embodiment, the animal is a mammal. In one embodiment, the mammal is a human.

In one embodiment of the present method, the determining comprises a technique selected from the group consisting of a Northern blot, hybridization to a nucleic acid microarray, and a reverse transcription-polymerase chain reaction (RT-PCR). In one embodiment, the RT-PCR is quantitative RT-PCR. In alternative embodiments, the determining is of the expression levels of at least two genes, of at least five genes, at least ten genes, at least twenty genes, at least twenty-five genes, or of all of the genes identified in SEQ ID NOs: 1-70.

In one embodiment of the present method, the comparing comprises: (a) establishing an average expression level for each gene in a population, wherein the population comprises statistically significant numbers of normal subjects and subjects that have one or more different autoimmune disorders; (b) assigning a first value to each gene for which the expression level in the subject is higher than the average expression level in the population and a second value to each gene for which the expression level in the subject is lower than the average expression level in the population; and (c) adding the values assigned in step (b) to arrive at a sum, wherein the sum is indicative of the presence or absence of an autoimmune disorder in the subject.

The presently claimed subject matter also provides a kit comprising a plurality of oligonucleotide primers and instructions for employing the plurality of oligonucleotide primers to determine the expression level of, in alternative embodiments, at least one, at least five, at least ten, at least twenty, at least thirty, or all of the genes represented by SEQ ID NOs: 1-70. In one embodiment, the kit further comprises oligonucleotide primers to determine the expression level of a control gene.

### BRIEF DESCRIPTION OF THE DRAWINGS

FIGS. 1A and 1B depict Cluster Analysis of Pre- and Post-Immune Data.

FIG. 1A depicts an unsupervised self-organizing map that compares individuals before immunization (CONTROL) or after immunization (IMM, days 6-9 postimmunization) with influenza antigen. In the upper panel of FIG. 1A, profiles from the analysis of all genes are depicted. In the lower panel of FIG. 1A, profiles after removal of invariant genes are depicted. Individuals (designated 11 through 18) are connected by brackets.

FIG. 1B depicts K-means analysis of the data set. In FIG. 1B, data are presented as the natural logarithm of the ratio of the experimental group indicated on the X-axis to the control group. Individual lines in the plot represent expression ratios of the individual genes over the time course.

FIGS. 2A and 2B depict a comparison of the immune and autoimmune classes by cluster analysis.

In FIG. 2A, the immune (6-8 days post-immunization), RA and SLE groups were analyzed using a hierarchical clustering algorithm (upper panel). The immune, MS, and type 1 diabetes groups were subjected to similar cluster analysis (lower panel).

In FIG. 2B, K-means analysis was used to identify two distinct clusters of genes that were uniformly over-expressed (left panel) or under-expressed (right panel) in all four autoimmune groups. Data are presented as the natural logarithm of the ratio of the immune group or each autoimmune group (type 1 diabetes, MS, RA, or SLE) to the control group.

FIGS. 3A and 3B depict the analysis of the most under- and over-expressed genes in the autoimmune population on an individual basis. Expression levels of the individual genes were compared among 10 control individuals (black solid bars) and 25 individuals with autoimmune disease (gray stippled bars).

FIG. 3A depicts the expression levels of the ten most over-expressed genes.

FIG. 3B depicts the expression levels of the ten most under-expressed genes.

FIG. 4 depicts the classification and predication of autoimmune disease. The score (Y-axis) is shown for each individual sample analyzed from the different populations (X-axis). P-values are depicted in the legend, which is repeated here as follows immune=0.9; SLE=1E-08; RA=4E-07; IDDM=1E-06; MS=1E-06; SLE(2)=8E-07; RA(2)=5E-07; and family=1E-06. The 35 genes employed to derive this score were as follows: TGM2, SSP29, TAF21, LLGL2, TNFAIP2, SIP1, BPHL, TP53, DIPA, ASL, GNB5, MAN1A1, R09503, LOC51643, BMP8, ORC1L, W04674, R94175, CDH1, SUDD, EPB72, CDKN1B, CASP6, TXK, MYO1C, LIF, HSJ2, BRCA1, GUCY1B3, AP3S2, N68565, SC65, UB32G2, SLC16A4, and MMP17.

### BRIEF DESCRIPTION OF THE SEQUENCE LISTING

SEQ ID NOs: 1 and 2 are the nucleic acid sequences of a partial cDNA and a full-length cDNA, respectively, corresponding to the human transglutaminase 2 (TGM2) gene (GenBank Accession Nos. AA156324 and NM_{—}004613).

SEQ ID NOs: 3 and 4 are the nucleic acid sequences of a partial cDNA and a full-length cDNA, respectively, corresponding to the human acidic (leucine-rich) nuclear phosphoprotein 32 family, member B (ANP32B, also called silver-stainable protein 29; SSP29) gene (GenBank Accession Nos. AA489201 and NM_{—}006401).

SEQ ID NOs: 5 and 6 are the nucleic acid sequences of a partial cDNA and a full-length cDNA, respectively, corresponding to the human TATA box binding protein (TBP)-associated factor 11 (TAF11) RNA polymerase II, 28 kilodalton (kDa) gene (TAF2I) (GenBank Accession Nos. N92711 and NM_{—}005643).

SEQ ID NOs: 7 and 8 are the nucleic acid sequences of a partial cDNA and a full-length cDNA, respectively, corresponding to the human lethal giant larvae homolog 2 (LLGL2) gene (GenBank Accession Nos. T40541 and NM_{—}004524).

SEQ ID NOs: 9 and 10 are the nucleic acid sequences of a partial cDNA and a full-length cDNA, respectively, corresponding to the human tumor necrosis factor, alpha-induced protein 2 (TNFAIP2) gene (GenBank Accession Nos. AA457114 and NM_{—}006291).

SEQ ID NOs: 11 and 12 are the nucleic acid sequences of a partial cDNA and a full-length cDNA, respectively, corresponding to the human survival of motor neuron protein interacting protein 1 (SIP1) gene (GenBank Accession Nos. N26026 and NM_{—}003616).

SEQ ID NOs: 13 and 14 are the nucleic acid sequences of a partial cDNA and a full-length cDNA, respectively, corresponding to the human biphenyl hydrolase-like (BPHL; serine hydrolase; breast epithelial mucin-associated antigen) gene (GenBank Accession Nos. AA171449 and NM_{—}004332).

SEQ ID NOs: 15 and 16 are the nucleic acid sequences of a partial cDNA and a full-length cDNA, respectively, corresponding to the human tumor protein p53 (TP53; Li-Fraumeni syndrome) gene (GenBank Accession Nos. R39356 and NM_{—}000546).

SEQ ID NOs: 17 and 18 are the nucleic acid sequences of a partial cDNA and a full-length cDNA, respectively, corresponding to the human hepatitis delta antigen-interacting protein A (DIPA) gene (GenBank Accession Nos. N94820 and NM_{—}006848).

SEQ ID NOs: 19 and 20 are the nucleic acid sequences of a partial cDNA and a full-length cDNA, respectively, corresponding to the human argininosuccinate lyase (ASL) gene (GenBank Accession Nos. AA486741 and NM_{—}000048).

SEQ ID NO: 21 and 22 are the nucleic acid sequences of a partial cDNA and a full-length cDNA, respectively, corresponding to the human gene identified as DKFZp586O1922 (GenBank Accession Nos. H08753 and AL117471).

SEQ ID NOs: 23 and 24 are the nucleic acid sequences of a partial cDNA and a full-length cDNA, respectively, corresponding to the human mannosidase, alpha, class 1A, member 1 (MAN1A1) gene (GenBank Accession Nos. T91261 and NM_{—}005907).

SEQ ID NO: 25 is a nucleic acid sequence of an expressed sequence tag (EST) designated R09503 in the GenBank database. This gene shows substantial homology to bases 106283 to 106592 of the BAC sequence from the SPG4 candidate region at 2p21-2p22 BAC 41M14 of library CITB_{—}978_SKB from human chromosome 2 (SEQ ID NO: 26; GenBank Accession Number AL121657.4).

SEQ ID NO: 27 is a nucleic acid sequence of a partial cDNA with GenBank Accession number AA130874. This gene shows substantial homology to the human CGI-119 gene (SEQ ID NO: 28; GenBank Accession Number NM_{—}016056).

SEQ ID NOs: 29 and 30 are the nucleic acid sequences of a partial cDNA and a full-length cDNA, respectively, corresponding to the human bone morphogenetic protein 8 (osteogenic protein 2; BMP8) gene (GenBank Accession Nos. AA779480 and NM_{—}001720).

SEQ ID NOs: 31 and 32 are the nucleic acid sequences of a partial cDNA and a full-length cDNA, respectively, corresponding to the human cytochrome b5 outer mitochondrial membrane precursor (CYB5-M) gene (GenBank Accession Nos. W04674 and NM_{—}030579.).

SEQ ID NOs: 33 and 34 are the nucleic acid sequences of a partial cDNA and a full-length cDNA, respectively, corresponding to the human origin recognition complex, subunit 1-like (ORC1L) gene (GenBank Accession Nos. R83277 and NM_{—}004153.).

SEQ ID NO: 35 is a nucleic acid sequence of an EST designated R94175 in the GenBank database. This EST shows substantial homology to bases 68656 to 68886 of BAC clone R-431H16 of library RPCI-11 from human chromosome 14 (SEQ ID NO: 36; GenBank Accession Number AL161665.5).

SEQ ID NOs: 37 and 38 are the nucleic acid sequences of a partial cDNA and a full-length cDNA, respectively, corresponding to the human cadherin 1, type 1, E-cadherin (epithelial; CDH1) gene (GenBank Accession Nos. H97778 and NM_{—}004360).

SEQ ID NOs: 39 and 40 are the nucleic acid sequences of a partial cDNA and a full-length cDNA, respectively, corresponding to the human sudD suppressor of bimD6 homolog (SUDD) from Aspergillus nidulans, transcript variant 1 gene (GenBank Accession Nos. T54144 and NM_{—}003831).

SEQ ID NOs: 41 and 42 are the nucleic acid sequences of a partial cDNA and a full-length cDNA, respectively, corresponding to the human stomatin (STOM; also called EPB72) gene (GenBank Accession Nos. R62817 and NM_{—}004099).

SEQ ID NOs: 43 and 44 are the nucleic acid sequences of a partial cDNA and a full-length cDNA, respectively, corresponding to the human cyclin-dependent kinase inhibitor 1B (CDKN1B) gene (GenBank Accession Nos. AA630082 and NM_{—}004064).

SEQ ID NOs: 45 and 46 are the nucleic acid sequences of a partial cDNA and a full-length cDNA, respectively, corresponding to the human caspase 6 (CASP6) gene (GenBank Accession Nos. W45688 and NM_{—}001226).

SEQ ID NOs: 47 and 48 are the nucleic acid sequences of a partial cDNA and a full-length cDNA, respectively, corresponding to the human TXK tyrosine kinase (TXK) gene (GenBank Accession Nos. H12312 and NM_{—}003328).

SEQ ID NOs: 49 and 50 are the nucleic acid sequences of a partial cDNA and a full-length cDNA, respectively, corresponding to the human myosin IC (MYO1C) gene (GenBank Accession Nos. M485871 and NM_{—}033375).

SEQ ID NOs: 51 and 52 are the nucleic acid sequences of a partial cDNA and a full-length cDNA, respectively, corresponding to the human leukemia inhibitory factor (LIF) gene (GenBank Accession Nos. AA026609 and NM_{—}002309).

SEQ ID NOs: 53 and 54 are the nucleic acid sequences of a partial cDNA and a full-length cDNA, respectively, corresponding to the human DnaJ homolog, subfamily A, member 1 (DNAJA1) gene (GenBank Accession Nos. R45428 and NM_{—}001539).

SEQ ID NOs: 55 and 56 are the nucleic acid sequences of a partial cDNA and a full-length cDNA, respectively, corresponding to the human breast cancer 1, early onset (BRCA1), transcript variant BRCA1 a gene (GenBank Accession Nos. H90415 and NM_{—}007294).

SEQ ID NOs: 57 and 58 are the nucleic acid sequences of a partial cDNA and a full-length cDNA, respectively, corresponding to the human guanylate cyclase 1, soluble, beta 3 (GUCY1B3) gene (GenBank Accession Nos. AA458785 and NM_{—}000857).

SEQ ID NOs: 59 and 60 are the nucleic acid sequences of a partial cDNA and a full-length cDNA, respectively, corresponding to the human adaptor-related protein complex 3, sigma 2 subunit (AP3S2) gene (GenBank Accession Nos. R33031 and NM_{—}005829).

SEQ ID NOs: 61 and 62 are the nucleic acid sequences of a partial cDNA and a full-length cDNA, respectively, corresponding to the human reticulon 4 (RTN4) gene, listed in the GenBank database at accession number N68565 (GenBank Accession Nos. N68565 and NM_{—}007008).

SEQ ID NOs: 63 and 64 are the nucleic acid sequences of a partial cDNA and a full-length cDNA, respectively, corresponding to the human 55 kDa nucleolar autoantigen similar to rat synaptonemal complex protein (SC65) gene (GenBank Accession Nos. W81191 and NM_{—}006455).

SEQ ID NOs: 65 and 66 are the nucleic acid sequences of a partial cDNA and a full-length cDNA, respectively, corresponding to the human ubiquitin-conjugating enzyme E2G 2 (UBC7 homolog, yeast; UBE2G2) gene (GenBank Accession Nos. AA443634 and NM_{—}003343).

SEQ ID NOs: 67 and 68 are the nucleic acid sequences of a partial cDNA and a full-length cDNA, respectively, corresponding to the human solute carrier family 16, member 4 (SLC16A4) gene (GenBank Accession Nos. R73608 and NM_{—}004696).

SEQ ID NO: 69 and 70 are the nucleic acid sequences of a partial cDNA and a full-length cDNA, respectively, corresponding to the human matrix metalloproteinase 17 (MMP17) gene (GenBank Accession Nos. R42600 and NM_{—}016155).

### DETAILED DESCRIPTION

The presently claimed subject matter relates to methods for detecting an autoimmune disorder in a subject by analyzing gene expression profiles for selected genes in biological samples isolated from the subject and comparing the gene expression profiles to standards. In one embodiment, the methods involve determining the expression levels of a set of genes expressed in peripheral blood mononuclear cells isolated from a subject suspected of having an autoimmune disease and comparing the expression levels of these genes with the levels of expression of these genes in normal subjects and subjects with confirmed autoimmune diseases. Using the methods of the presently claimed subject matter, it is possible to determine whether or not a subject has an autoimmune disease (for example, rheumatoid arthritis, systemic lupus erythematosus, multiple sclerosis, and/or type 1 (insulin-dependent) diabetes) or whether the subject does not have autoimmune disease.

In determining whether or not a subject has an autoimmune disease, the expression levels of many genes can be analyzed simultaneously using microarrays or membrane-based filter arrays. A representative filter array is the GF211 Human “Named Genes” GENEFILTERS® Microarrays Release 1 (available from RESGEN™, a division of Invitrogen Corporation, Carlsbad, Calif., United States of America), although other arrays can also be used. Using the GF211 array, it is possible to determine the expression levels of over 4000 genes simultaneously in a biological sample. Additionally, the presence on the GF211 filter of certain “housekeeping” genes allows for the comparison of data from experiment to experiment. This facilitates the comparison of newly obtained data to a standard (e.g. a previously generated standard).

I. Definitions

While the following terms are believed to be well understood by one of ordinary skill in the art, the following definitions are set forth to facilitate explanation of the presently claimed subject matter.

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, in another example ±5%, in another example ±1%, and in still another example ±0.1% from the specified amount, as such variations are appropriate to perform the disclosed method.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant.

In one example, a p-value less than or equal to 0.05, in another example less than 0.01, in another example less than 0.005, and in yet another example less than 0.001, are regarded as significant.

I.A. Nucleic acids

The nucleic acid molecules employed in accordance with the presently claimed subject matter include any nucleic acid molecule for which expression is desired to be assessed in evaluating the presence or absence of an autoimmune disease. Representative nucleic acid molecules include, but are not limited to, the isolated nucleic acid molecules of any one of SEQ ID NOs: 1-70, complementary DNA molecules, sequences having 80% identity as disclosed herein to any one of SEQ ID NOs: 1-70, sequences capable of hybridizing to any one of SEQ ID NOs: 1-70 under conditions disclosed herein, and corresponding RNA molecules.

As used herein, “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can comprise monomers that are naturally occurring nucleotides (such as deoxyribonucleotides and ribonucleotides), or analogs of naturally occurring nucleotides (e.g., α-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups. Sugars can also be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of phosphodiester bonds. Analogs of phosphodiester linkages include phosphorothioate, phosphorodithioate, phosphoroselenoate, phosphorodiselenoate, phosphoroanilothioate, phosphoranilidate, phosphoramidate, and the like.

Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism. In one embodiment, a nucleic acid is derived from a biological sample isolated from a subject.

The term “subsequence” refers to a sequence of nucleic acids that comprises a part of a longer nucleic acid sequence. An exemplary subsequence is a probe, or a primer. The term “primer” as used herein refers to a contiguous sequence comprising in one example about 8 or more deoxyribonucleotides or ribonucleotides, in another example 10-20 nucleotides, and in yet another example 20-30 nucleotides of a selected nucleic acid molecule. The primers disclosed herein encompass oligonucleotides of sufficient length and appropriate sequence so as to provide initiation of polymerization on a target nucleic acid molecule.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

As used herein, the phrases “open reading frame” and “ORF” are given their common meaning and refer to a contiguous series of deoxyribonucleotides or ribonucleotides that encode a polypeptide or a fragment of a polypeptide. In an organism that splices precursor RNAs to form mRNAs, the ORF will be discontinuous in the genome. Splicing produces a continuous ORF that can be translated to produce a polypeptide. In a full-length cDNA, the complete ORF includes those nucleic acid sequences beginning with the start codon and ending with the stop codon. In a cDNA molecule that is not full-length, the ORF includes those nucleic acid sequences present in the non-full-length cDNA that are included within the complete ORF of the corresponding full-length cDNA.

As used herein, the phrase “coding sequence” is used interchangeably with “open reading frame” and “ORF” and refers to a nucleic acid sequence that is transcribed into RNA including, but not limited to mRNA, rRNA, tRNA, snRNA, sense RNA, or antisense RNA. The RNA can then be translated in vitro or in vivo to produce a protein.

The terms “complementary” and “complementary sequences”, as used herein, refer to two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth herein, or is defined as being capable of hybridizing to the nucleic acid segment in question under relatively stringent conditions such as those described herein. In one embodiment, a complementary sequence is at least 80% complementary to the nucleotide sequence with which is it capable of pairing. In another embodiment, a complementary sequence is at least 85% complementary to the nucleotide sequence with which is it capable of pairing. In another embodiment, a complementary sequence is at least 90% complementary to the nucleotide sequence with which is it capable of pairing. In another embodiment, a complementary sequence is at least 95% complementary to the nucleotide sequence with which is it capable of pairing. In another embodiment, a complementary sequence is at least 98% complementary to the nucleotide sequence with which is it capable of pairing. In another embodiment, a complementary sequence is at least 99% complementary to the nucleotide sequence with which is it capable of pairing. In still another embodiment, a complementary sequence is at 100% complementary to the nucleotide sequence with which is it capable of pairing. A particular example of a complementary nucleic acid segment is an antisense oligonucleotide.

The term “gene” refers broadly to any segment of DNA associated with a biological function. A gene encompasses sequences including, but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including isolation or cloning from a biological sample, synthesis based on known or predicted sequence information, and recombinant derivation of an existing sequence.

As used herein, the terms “known gene” and “reference gene” are used interchangeably and refer to nucleic acid sequences that can be identified as corresponding to a particular expressed sequence tag (EST), partial cDNA, full-length cDNA, or gene. In one embodiment, a reference gene is a gene, a cDNA, or an EST for which the nucleic acid sequence has been determined (i.e. is known). In another embodiment, a reference gene is represented by one of the nucleic acid sequences disclosed in SEQ ID NOs: 1-70. In another embodiment, a reference gene is represented by a nucleic acid sequence complementary to one of the nucleic acid sequences disclosed in SEQ ID NOs: 1-70. In another embodiment, a reference gene is represented by a nucleic acid sequence having 80% identity to any one of SEQ ID NOs: 1-70. In another embodiment, a reference gene is represented by a nucleic acid sequence capable of hybridizing to any one of SEQ ID NOs: 1-70 under conditions disclosed herein. In another embodiment, a reference gene is represented by an RNA molecule corresponding to any one of SEQ ID NOs: 1-70. In another embodiment, a reference gene is represented by a nucleic acid sequence present on an array.

As used herein, the terms “corresponding to” and “representing”, “represented by” and grammatical derivatives thereof, when used in the context of a nucleic acid sequence corresponding to or representing a gene, refers to a nucleic acid sequence that results from transcription, reverse transcription, or replication from a particular genetic locus, gene, or gene product (for example, an mRNA). In other words, an EST, partial cDNA, or full-length cDNA corresponding to a particular reference gene is a nucleic acid sequence that one of ordinary skill in the art would recognize as being a product of either transcription or replication of that reference gene (for example, a product produced by transcription of the reference gene). One of ordinary skill in the art would understand that the EST, partial cDNA, or full- length cDNA itself is produced by in vitro manipulation to convert the mRNA into an EST or cDNA, for example by reverse transcription of an isolated RNA molecule that was transcribed from the reference gene. One of ordinary skill in the art will also understand that the product of a reverse transcription is a double-stranded DNA molecule, and that a given strand of that double-stranded molecule can embody either the coding strand or the non-coding strand of the gene. The sequences presented in the Sequence Listing are single-stranded, however, and it is to be understood that the presently claimed subject matter is intended to encompass the genes represented by the sequences presented in SEQ ID NOs: 1-70, including the specific sequences set forth as well as the reverse/complement of each of these sequences.

A known gene and/or reference gene also includes, but is not limited to those genes that have been identified as being differentially expressed in autoimmune patients versus normal patients, such as but not limited to those set forth in Table 1. A reference gene is also intended to include nucleic acid sequences that substantially hybridize to one of such genes, including but not limited to one of the nucleic acid sequences disclosed in SEQ ID NOs: 1-70. As such, a reference gene includes a nucleic acid sequence that has one or more polymorphisms such that while the particular nucleic acid sequence might diverge somewhat from one of such genes, including but not limited to one of those disclosed in SEQ ID NOs: 1-70, one of ordinary skill in the art would nonetheless recognize the particular nucleic acid sequence as corresponding to a gene represented by one of such genes, including but not limited to one of the sequences disclosed in SEQ ID NOs: 1-70. For example, the GenBank database has at least three accession numbers that are identified as corresponding to the human breast cancer 1, early onset (BRCA1) mRNA. These three represent transcript variants a, a′, and b, and have accession numbers NM_{—}007294, NM_{—}007296, and NM_{—}007295, respectively. It is understood that the presently claimed subject matter, which identifies NM_{—}007294 as SEQ ID NO: 56, also encompasses the other transcript variants.

In the context of the presently claimed subject matter, a reference gene is also intended to include nucleic acid sequences that substantially hybridize to a nucleic acid corresponding to a gene represented by one of the nucleic acid sequences disclosed in SEQ ID NOs: 1-70. As such, a reference gene includes a nucleic acid sequence that has one or more polymorphisms such that while the particular nucleic acid sequence might diverge somewhat from those disclosed in SEQ ID NOs: 1-70, one of ordinary skill in the art would nonetheless recognize the particular nucleic acid sequence as corresponding to a gene represented by one of the sequences disclosed in SEQ ID NOs: 1-70.

The term “gene expression” generally refers to the cellular processes by which a biologically active polypeptide is produced from a DNA sequence. Generally, gene expression comprises the processes of transcription and translation, along with those modifications that normally occur in the cell to modify the newly translated protein to an active form and to direct it to its proper subcellular or extracellular location.

The terms “gene expression level” and “expression level” as used herein refer to an amount of gene-specific RNA or polypeptide that is present in a biological sample. When used in relation to an RNA molecule, the term “abundance” can be used interchangeably with the terms “gene expression level” and “expression level”. While an expression level can be expressed in standard units such as “transcripts per cell” for RNA or “nanograms per microgram tissue” for RNA or a polypeptide, it is not necessary that expression level be defined as such. Alternatively, relative units can be employed to describe an expression level. For example, when the assay has an internal control (referred to herein as a “control gene”), which can be, for example, a known quantity of a nucleic acid derived from a gene for which the expression level is either known or can be accurately determined, unknown expression levels of other genes can be compared to the known internal control. More specifically, when the assay involves hybridizing labeled total RNA to a solid support comprising a known amount of nucleic acid derived from known genes, an appropriate internal control could be a housekeeping gene (e.g. glucose-6-phosphate dehydrogenase or elongation factor-1), a ideal housekeeping gene being defined as a gene for which the expression level in all cell types and under all conditions is the same. Use of such an internal control allows relative expression levels to be determined (e.g. relative to the expression of the housekeeping gene) both for the nucleic acids present on the solid support and also between different experiments using the same solid support. This discrete expression level can then be normalized to a value relative to the expression level of the control gene (for example, a housekeeping gene).

As used herein, the term “normalized”, and grammatical derivatives thereof, refers to a manipulation of discrete expression level data wherein the expression level of a reference gene is expressed relative to the expression level of a control gene. For example, the expression level of the control gene can be set at 1, and the expression levels of all reference genes can be expressed in units relative to the expression of the control gene.

The term “average expression level” as used herein refers to the mean expression level, in whatever units are chosen, of a gene in a particular biological sample of a population. To determine an average expression level, a population is defined, and the expression level of the gene in that population is determined for each member of the population by analyzing the same biological sample from each member of the population. The determined expression levels are then added together, and the sum is divided by the number of members in the population.

The term “average expression level” is also used to refer to a calculated value that can be used to compare two populations. For example, the average expression level in a population consisting of all patients regardless of autoimmune disease status can be calculated using the method above for a population that consists of statistically significant numbers of patients with and without autoimmune disease (the latter can also be referred to as the “unaffected subpopulation”). However, when the population is made up of unequal numbers of patients with and without autoimmune disease, the calculated value for all genes differentially expressed in these two subpopulations will likely be skewed towards the expression level determined for the subpopulation having the greater number of members. In order to remove this skewing effect, the average expression level in the described population can also be calculated by: (a) determining the average expression level of a gene in the autoimmune patient subpopulation; (b) determining the average expression level of the same gene in the unaffected subpopulation; (c) adding the two determined values together; and (d) dividing the sum of the two determined values by 2 to achieve a value: this value also being defined herein as an “average expression level”.

Once an expression level is determined for a gene, a profile can be created. As used herein, the term “profile” refers to a repository of the expression level data that can be used to compare the expression levels of different genes among various subjects. For example, for a given subject, the term “profile” can encompass the expression levels of all genes detected in whatever units (as described herein above) are chosen.

The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison. In one embodiment, a standard is prepared by determining the average expression level of a gene in a normal population, a normal population being defined as subjects that do not have autoimmune disease. In another embodiment, a standard is prepared by determining the average expression level of a gene in a population of subjects that have an autoimmune disease (for example, RA, MS, IDDM, and/or SLE). In a third embodiment, a standard is prepared by determining the average expression level of a gene in the population as a whole (i.e. subjects are grouped together irrespective of autoimmune disease status). In yet another embodiment, a standard is prepared by determining the average expression level of a gene in a normal population, the average expression level of a gene in an autoimmune population, adding those two values, and dividing the sum by two to determine the midpoint of the average expression in these populations. In this latter embodiment, a profile for a “new” subject can be compared to the standard, and the profile can further comprise data indicating whether for each gene, the expression level in the new subject is higher or lower than the expression level of that gene in the standard. For example, a new subject's profile can comprise a score of “1” for each gene for which the expression in the subject is higher than in the standard, and a score of “0” for each gene for which the expression in the subject is lower than in the standard. In this way, a profile can comprise an overall “score”, the score being defined as the sum total of all the ones and zeroes present in the profile. These scores can then be used to predict the presence or absence of autoimmune disease in the new subject. It is understood that the use of 1s and 0s is exemplary only, and any convenient value can be assigned in the practice of the methods of the presently claimed subject matter.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as, for example, in a host cell transformed with a vector comprising the DNA molecule.

The phrases “percent identity” and “percent identical,” in the context of two nucleic acid or protein sequences, refer to two or more sequences or subsequences that have in one embodiment at least 60%, in another embodiment at least 70%, in another embodiment at least 80%, in another embodiment at least 85%, in another embodiment at least 90%, in another embodiment at least 95%, in another embodiment at least 98%, and in yet another embodiment at least 99% nucleotide or amino acid residue identity, when compared and aligned for maximum correspondence, as measured using one of the following sequence comparison algorithms or by visual inspection. The percent identity exists in one embodiment over a region of the sequences that is at least about 50 residues in length, in another embodiment over a region of at least about 100 residues, and in still another embodiment the percent identity exists over at least about 150 residues. In yet another embodiment, the percent identity exists over the entire length of a given region, such as a coding region. In one embodiment, a nucleic acid is at least 80% identical to one of SEQ ID NOs: 1-70.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, FASTA, and TFASTA in the GCG Wisconsin Package, available from Accelrys, Inc., San Diego, Calif., United States of America), or by visual inspection. See generally, Ausubel et al., 1994.

One example of an algorithm that is suitable for determining percent sequence identity and sequence similarity is the BLAST algorithm, which is described in Altschul et al., 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff 1989.

In addition to calculating percent sequence identity, the BLAST algorithm also performs a statistical analysis of the similarity between two sequences. See e.g., Karlin & Altschul 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The term “substantially identical”, in the context of two nucleotide sequences, refers to two or more sequences or subsequences that have in one embodiment at least about 80% nucleotide identity, in another embodiment at least about 85% nucleotide identity, in another embodiment at least about 90% nucleotide identity, in another embodiment at least about 95% nucleotide identity, in another embodiment at least about 98% nucleotide identity, and in yet another embodiment at least about 99% nucleotide identity, when compared and aligned for maximum correspondence, as measured using one of the following sequence comparison algorithms or by visual inspection. In one example, the substantial identity exists in nucleotide sequences of at least 50 residues, in another example in nucleotide sequence of at least about 100 residues, in another example in nucleotide sequences of at least about 150 residues, and in yet another example in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair. Nonetheless, one of ordinary skill in the art would recognize that the polymorphic sequences correspond to the same gene. For example, SEQ ID NO: 1-70 is an EST derived from the human TP53 gene. The human TP53 complete cDNA sequence (SEQ ID NO: 16) is present in the GenBank database under Accession Number NM_{—}000546, and according to the description presented therein, the TP53 gene is characterized by polymorphisms at nucleotide positions 390, 466, 1470, 1927, 1950, 1976, 1977, 2075, 2076, 2497, and 2498. Nucleic acid sequences comprising any or all of these polymorphisms are substantially identical to SEQ ID NO: 1-70, and thus are intended to be encompassed within the claimed subject matter.

Another indication that two nucleotide sequences are substantially identical is that the two molecules specifically or substantially hybridize to each other under stringent conditions. In the context of nucleic acid hybridization, two nucleic acid sequences being compared can be designated a “probe sequence” and a “target sequence”. A “probe sequence” is a reference nucleic acid molecule, and a “target sequence” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in one embodiment at least an about 14 to 40 nucleotide sequence of a nucleic acid molecule of the presently claimed subject matter. In one example, probes comprise 14 to 20 nucleotides, or even longer where desired, such as 30, 40, 50, 60, 100, 200, 300, or 500 nucleotides or up to the full length of any of the genes represented by SEQ ID NOs: 1-70. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (see Sambrook and Russell, 2001, for a description of SSC buffer). Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently claimed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% SDS, 0.5M NaPO₄, 1 mm EDTA at 50° C. followed by washing in 1×SSC, 0.1% SDS at 50° C.; in another example, a probe and target sequence hybridize in 7% SDS, 0.5M NaPO₄, 1 mm EDTA at 50° C. followed by washing in 0.5×SSC, 0.1% SDS at 50° C.; in another example, a probe and target sequence hybridize in 7% SDS, 0.5M NaPO₄, 1 mm EDTA at 50° C. followed by washing in 0.1×SSC, 0.1% SDS at 50° C.; in yet another example, a probe and target sequence hybridize in 7% SDS, 0.5M NaPO₄, 1 mm EDTA at 50° C. followed by washing in 0.1×SSC, 0.1% SDS at 65° C. In one embodiment, hybridization conditions comprise hybridization in a roller tube for at least 12 hours at 42° C.

Pre-made hybridization solutions are also commercially available from various suppliers. In one embodiment, a hybridization solution comprises MICROHYB™ (RESGEN™), and in another embodiment a hybridization solution comprises MICROHYB™ further comprising 5.0 μg COT-1® DNA (Invitrogen Corporation, Carlsbad, Calif., United States of America) and 5.0 μg poly-dA. In one embodiment, post-hybridization wash conditions comprise two washes in 2×SSC/1% SDS at 50° C. for 20 minutes each followed by a third wash in 0.5×SSC/1% SDS at 55° C. for 15 minutes.

As used herein, the term “purified”, when applied to a nucleic acid or protein, denotes that the nucleic acid or protein is essentially free of other cellular components with which it is associated in the natural state. It can be in a homogeneous state although it also can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is in one embodiment at least about 50% pure, in another embodiment at least about 85% pure, and in still another embodiment at least about 99% pure.

I.B. Biological Samples

The presently claimed subject matter provides methods that can be used to detect the expression level of a gene in a biological sample. The term “biological sample” as used herein refers to a sample that comprises a biomolecule that permits the expression level of a gene to be determined. Representative biomolecules include, but are not limited to total RNA, mRNA, and polypeptides. As such, a biological sample can comprise a cell or a group of cells. Any cell or group of cells can be used with the methods of the presently claimed subject matter, although cell-types and organs that would be predicted to show differential gene expression in subjects with autoimmune disease versus normal subjects are best suited. In one embodiment, gene expression levels are determined where the biological sample comprises PBMCs. In one embodiment, the biological sample comprises one or more of the constituent cell types that make up a PBMC preparation, including but not limited to T cells, B cells, monocytes, and NK/NKT cells. A representative PMBC preparation can comprise about 75% T cells, about 5% to about 10% B cells, about 5% to about 10% monocytes, and a small percentage of NK/NKT cells. In another embodiment, the biological sample comprises epithelial cells, such as cheek epithelial cells. Also encompassed within the phrase “biological sample” are biomolecules that are derived from a cell or group of cells that permit gene expression levels to be determined, e.g. nucleic acids and polypeptides.

The expression level of the gene can be determined using molecular biology techniques that are well known in the art. For example, if the expression level is to be determined by analyzing RNA isolated from the biological sample, techniques for determining the expression level include, but are not limited to Northern blotting, quantitative PCR, and the use of nucleic acid arrays and microarrays.

In one embodiment, the expression level of a gene is determined by hybridizing ³³P-labeled cDNA generated from total RNA isolated from a biological sample to one or more DNA sequences representing one or more genes that has been affixed to a solid support, e.g. a membrane. When a membrane comprises nucleic acids representing many genes (including internal controls), the relative expression level of many genes can be determined. The presence of internal control sequences on the membrane also allows experiment-to-experiment variations to be detected, yielding a strategy whereby the raw expression data derived from each experiment can be compared from experiment-to-experiment.

Alternatively, gene expression can be determined by analyzing protein levels in a biological sample using antibodies. Representative antibody-based techniques include, but are not limited to immunoprecipitation, Western blotting, and the use of immunoaffinity columns.

The term “subject” as used herein refers to any vertebrate species. The methods of the presently claimed subject matter are particularly useful in the diagnosis of warm-blooded vertebrates. Thus, the presently claimed subject matter concerns mammals. More particularly contemplated is the diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the diagnosis of autoimmune disease in livestock, including, but not limited to domesticated swine (pigs and hogs), ruminants, horses, poultry, and the like.

II. Isolation and Analysis of Nucleic Acids

II.A. Enrichment of Nucleic Acids

The presently claimed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson 1994; Millar et al., 1995), filtration columns (Bej et a/., 1991), or immunomagnetic beads (Albert et al., 1992; Chiodi et al., 1992). Such approaches can significantly increase the sensitivity of subsequent detection methods.

As one example, SEPHADEX® matrix (Sigma, St. Louis, Mo., United States of America) is a matrix of diatomaceous earth and glass suspended in a solution of chaotropic agents and has been used to bind nucleic acid material (Boom et al., 1990; Buffone et al., 1991). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al., 1992).

II.B. Nucleic Acid Isolation

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

When total RNA or purified mRNA is selected as a biological sample, the disclosed method enables an assessment of a level of gene expression. For example, detecting a level of gene expression in a biological sample can comprise determination of the abundance of a given mRNA species in the biological sample.

RNA isolation methods are known to one of skill in the art. See Albert et al., 1992; Busch et al., 1992; Hamel et al., 1995; Herrewegh et al., 1995; Izraeli et al., 1991; McCaustland et al., 1991; Natarajan et al., 1994; Rupp et al., 1988; Tanaka et al., 1994; Vankerckhoven et al., 1994. A representative procedure for RNA isolation from a biological sample is set forth in Example 2.

Simple and semi-automated extraction methods can also be used for nucleic acid isolation, including for example, the SPLIT SECOND™ system (Boehringer Mannheim, Indianapolis, Ind., United States of America), the TRIZOL™ Reagent system (Life Technologies, Gaithersburg, Md., United States of America), and the FASTPREP™ system (Bio 101, La Jolla, Calif., United States of America). See also Paladichuk 1999.

Nucleic acids that are used for subsequent amplification and labeling can be analytically pure as determined by spectrophotometric measurements or by visual inspection following electrophoretic resolution. The nucleic acid sample can be free of contaminants such as polysaccharides, proteins, and inhibitors of enzyme reactions. When an RNA sample is intended for use as probe, it can be free of nuclease contamination. Contaminants and inhibitors can be removed or substantially reduced using resins for DNA extraction (e.g., CHELEX™ 100 from BioRad Laboratories, Hercules, Calif., United States of America) or by standard phenol extraction and ethanol precipitation. Isolated nucleic acids can optionally be fragmented by restriction enzyme digestion or shearing prior to amplification.

II.C. (PCR Amplification of Nucleic Acids

The terms “template nucleic acid” and “target nucleic acid” as used herein each refers to nucleic acids isolated from a biological sample as described herein above. The terms “template nucleic acid pool”, “template pool”, “target nucleic acid pool”, and “target pool” each refers to an amplified sample of “template nucleic acid”. Thus, a target pool comprises amplicons generated by performing an amplification reaction using the template nucleic acid. In one embodiment, a target pool is amplified using a random amplification procedure as described herein.

The term “target-specific primer” refers to a primer that hybridizes selectively and predictably to a target sequence, for example a sequence that shows differential expression in a patient with an autoimmune disease relative to a normal patient, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not designed for complementarity to a nucleotide sequence of the target-specific probe. The term “random primer” encompasses selection of an arbitrary sequence having increased probability to be efficiently utilized in an amplification reaction. For example, the Random Oligonucleotide Construction Kit (ROCK; available from http://www.sru.edu/depts/artsci/bio/ROCK.htm) is a macro-based program that facilitates the generation and analysis of random oligonucleotide primers (Strain & Chmielewski 2001). Representative primers include, but are not limited to random hexamers and rapid amplification of polymorphic DNA (RAPD)-type primers as described in Williams et al., 1990.

A random primer can also be degenerate or partially degenerate as described in Telenius et al., 1992. Briefly, degeneracy can be introduced by selection of alternate oligonucleotide sequences that can encode a same amino acid sequence.

In one embodiment, random primers can be prepared by shearing or digesting a portion of the template nucleic acid sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor is a heterologous primer. Representative heterologous primers can optionally include a poly(dT) primer, a poly(T) primer, or as appropriate, a poly(dA) primer or a poly(A) primer.

The term “primer” as used herein refers to a contiguous sequence comprising in one embodiment about 6 or more nucleotides, in another embodiment about 10-20 nucleotides (e.g. 15-mer), and in still another embodiment about 20-30 nucleotides (e.g. a 22-mer). Primers used to perform the method of the presently claimed subject matter encompass oligonucleotides of sufficient length and appropriate sequence so as to provide initiation of polymerization on a nucleic acid molecule.

II.C.1. Quantitative RT-PCR

In one embodiment of the presently claimed subject matter, the abundance of specific mRNA species present in a biological sample (for example, mRNA extracted from peripheral blood mononuclear cells) is assessed by quantitative RT-PCR. In this embodiment, standard molecular biological techniques are used in conjunction with specific PCR primers to quantitatively amplify those mRNA molecules corresponding to the genes of interest. Methods for designing specific PCR primers and for performing quantitative amplification of nucleic acids including mRNA are well known in the art. See e.g. Sambrook & Russell, 2001; Vandesompele et al., 2002; Joyce 2002.

II.C.2. Amplified Antisense RNA (aaRNA)

Several procedures have been developed specifically for random amplification of RNA, including but not limited to Amplified Antisense RNA (aaRNA) and Global RNA Amplification, also described further herein below. A population of RNA can be amplified using a technique referred to as Amplified Antisense RNA (aaRNA). See Van Gelder et al., 1990; Wang et al., 2000. Briefly, an oligo(dT) primer is synthesized such that the 5′ end of the primer includes a T7 RNA polymerase promoter. This oligonucleotide can be used to prime the poly(A)⁺ mRNA population to generate cDNA. Following first strand cDNA synthesis, second strand cDNA is generated using RNA nicking and priming (Sambrook & Russell 2001). The resulting cDNA is treated briefly with S1 nuclease and blunt-ended with T4 DNA polymerase. The cDNA is then used as a template for transcription-based amplification using the T7 RNA polymerase promoter to direct RNA synthesis.

Eberwine et al. adapted the aaRNA procedure for in situ random amplification of RNA followed by target-specific amplification. The successful amplification of under represented transcripts suggests that the pool of transcripts amplified by aaRNA is representative of the initial mRNA population (Eberwine et al., 1992).

II.C.3. Global RNA Amplification

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the presently claimed subject matter, any one of the above-mentioned PCR techniques or related techniques can be employed to perform the step of amplifying the nucleic acid sample. In addition, such methods can be optimized for amplification of a particular subset of nucleic acid (e.g., specific mRNA molecules versus total mRNA), and representative optimization criteria and related guidance can be found in the art. See Cha & Thilly 1993; Linz et al., 1990; Robertson & Walsh-Weller 1998; Roux 1995; Williams 1989; McPherson et al., 1995.

II.C.4. Kits for Gene Expression Analysis

The presently claimed subject matter also provides for kits comprising a plurality of oligonucleotide primers that can be used in the methods of the presently claimed subject matter to assess gene expression levels of genes of interest. In non-limiting embodiments, the kit can comprise oligonucleotide primers designed to be used to determine the expression level of one or more (e.g. 1, 5, 10, 20, 30, or all) of the genes set forth in SEQ ID NOs: 1-70. Additionally, the kit can comprise instructions for using the primers, including but not limited to information regarding proper reaction conditions and the sizes of the expected amplified fragments.

III. Nucleic Acid Labeling

In one embodiment, the expression level of a gene in a biological sample is determined by hybridizing total RNA isolated from the biological sample to an array containing known quantities of nucleic acid sequences corresponding to known genes. For example, the array can comprise single-stranded nucleic acids (also referred to herein as “probes” and/or “probe sets”) in known amounts for specific genes, which can then be hybridized to nucleic acids isolated from the biological sample. The array can be set up such that the nucleic acids are present on a solid support in such a manner as to allow the identification of those genes on the array to which the total RNA hybridizes. In this embodiment, the total RNA is hybridized to the array, and the genes to which the total RNA hybridizes are detected using standard techniques. In one embodiment of the presently claimed subject matter, the amplified nucleic acids are labeled with a radioactive nucleotide prior to hybridization to the array, and the genes on the array to which the RNA hybridizes are detected by autoradiography or phosphorimage analysis.

Alternatively, nucleic acids isolated from a biological sample are hybridized with a set of probes without prior labeling of the nucleic acids. For example, unlabeled total RNA isolated from the biological sample can be detected by hybridization to one or more labeled probes, the labeled probes being specific for those genes found to be useful in the methods of the presently claimed subject matter (e.g. those genes represented by SEQ ID NOs: 1-70). In another embodiment, both the nucleic acids and the one or more probes include a label, wherein the proximity of the labels following hybridization enables detection. An exemplary procedure using nucleic acids labeled with chromophores and fluorophores to generate detectable photonic structures is described in U.S. Pat. No. 6,162,603.

The nucleic acids or probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic (e.g. ³²P, ³³P, or ³⁵S) or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to fluorescein isothiocyanate (FITC), FLUOR X™, ALEXA FLUOR® 488, OREGON GREEN® 488, 6-JOE (6-carboxy-4′,5′-dichloro-2′, 7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech, Piscataway, N.J., United States of America, or from Molecular Probes Inc., Eugene, Oreg., United States of America). Fluorescent tags also include sulfonated cyanine dyes (available from Li-Cor, Inc., Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz 1996; Schena et al., 1995; Schena et al., 1996; Shalon et al., 1996; Shoemaker et al., 1996; Wang et al., 1998. A representative procedure is set forth herein as Example 6.

Indirect labeling techniques can also be used in accordance with the methods of the presently claimed subject matter, and in some cases, can facilitate detection of rare target sequences by amplifying the label during the detection step. Indirect labeling involves incorporation of epitopes, including recognition sites for restriction endonucleases, into amplified nucleic acids prior to hybridization with a set of probes. Following hybridization, a protein that binds the epitope is used to detect the epitope tag.

In one embodiment, a biotinylated nucleotide can be included in the amplification reactions to produce a biotin-labeled nucleic acid sample. Following hybridization of the biotin-labeled sample with probes as described herein, the label can be detected by binding of an avidin-conjugated fluorophore, for example streptavidin-phycoerythrin, to the biotin label. Alternatively, the label can be detected by binding of an avidin-horseradish peroxidase (HRP) streptavidin conjugate, followed by colorimetric detection of an HRP enzymatic product.

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner 1995). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in various hybridization assays, and that optimal labeling can be unique to each label type.

IV. Microarrays

In one embodiment of the presently claimed subject matter, nucleic acids isolated from a biological sample are hybridized to a microarray, wherein the microarray comprises nucleic acids corresponding to those genes to be tested as well as internal control genes. The genes are immobilized on a solid support, such that each position on the support identifies a particular gene. Solid supports include, but are not limited to nitrocellulose and nylon membranes. Solid supports can also be glass or silicon-based (i.e. gene “chips”). Any solid support can be used in the methods of the presently claimed subject matter, so long as the support provides a substrate for the localization of a known amount of a nucleic acid in a specific position that can be identified subsequent to the hybridization and detection steps. In one embodiment, a microarray comprises a nylon membrane (for example, the GF211 Human “Named Genes” GENEFILTERS® Microarrays Release 1 available from RESGEN™).

A microarray can be assembled using any suitable method known to one of skill in the art, and any one microarray configuration or method of construction is not considered to be a limitation of the presently claimed subject matter. Representative microarray formats that can be used in accordance with the methods of the presently claimed subject matter are described herein below.

IV.A. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include, but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman, Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al., 1997; Yershov et al., 1996). A BIOCHIP ARRAYER™ dispenser (Packard Instrument Company, Meriden, Conn., United States of America) can effectively dispense probes onto membranes such that the spot size is consistent among spots whether one, two, or four droplets were dispensed per spot (Englert 2000). The array can also comprise a dot blot or a slot blot.

A microarray substrate for use in accordance with the methods of the presently claimed subject matter can have either a two-dimensional (planar) or a three-dimensional (non-planar) configuration. An exemplary three-dimensional microarray is the FLOW-THRU™ chip (Gene Logic, Inc., Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al., 1998; Steel et al., 2000.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767.

IV.B. Surface Chemistry

The particular surface chemistry employed is inherent in the microarray substrate and substrate preparation. Immobilization of nucleic acids probes post-synthesis can be accomplished by various approaches, including adsorption, entrapment, and covalent attachment. Preferably, the binding technique does not disrupt the activity of the probe.

For substantially permanent immobilization, covalent attachment is preferred. Since few organic functional groups react with an activated silica surface, an intermediate layer is advisable for substantially permanent probe immobilization. Functionalized organosilanes can be used as such an intermediate layer on glass and silicon substrates (Liu & Hlady 1996; Shriver-Lake 1998). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized in Hermanson 1990. A representative protocol for covalent attachment of DNA to silicon wafers is described in O'Donnell et al., 1997.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner 1997; Schena et al., 1995). See also Worley et al., 2000.

For attachment of probes greater than about 300 base pairs, noncovalent binding is suitable. A representative technique for noncovalent linkage involves use of sodium isothiocyanate (NaSCN) in the spotting solution, as described in Example 7. When using this method, amino-silanized slides can be used since this coating improves nucleic acid binding when compared to bare glass. This method works well for spotting applications that use about 100 ng/μl (Worley et al., 2000).

In the case of nitrocellulose or nylon membranes, the chemistry of nucleic acid binding to these membranes has been well characterized (Southern 1975; Sambrook & Russell 2001). One-such nylon filter array is the GF211 Human “Named Genes” GENEFILTERS® Microarrays Release 1 (available from RESGEN™, a division of Invitrogen Corporation, Calsbad, Calif., United States of America), although other arrays can also be used.

IV.C. Arraying Techniques

A microarray for the detection of gene expression levels in a biological sample can be constructed using any one of several methods available in the art including, but not limited to photolithographic and microfluidic methods, further described herein below. In one embodiment, the method of construction is flexible, such that a microarray can be tailored for a particular purpose.

As is standard in the art, a technique for making a microarray should create consistent and reproducible spots. Each spot can be uniform, and appropriately spaced away from other spots within the configuration. A solid support for use in the presently claimed subject matter comprises in one embodiment about 10 or more spots, in another embodiment about 100 or more spots, in another embodiment about 1,000 or more spots, and in still another embodiment about 10,000 or more spots. In one embodiment, the volume deposited per spot is about 10 picoliters to about 10 nanoliters, and in another embodiment about 50 picoliters to about 500 picoliters. The diameter of a spot is in one embodiment about 50 μm to about 1000 μm, and in another embodiment about 100 μm to about 250 μm.

Light-directed synthesis. This technique was developed by Fodor et al. (Fodor et al., 1991; Fodor et al., 1993; U.S. Pat. No. 5,445,934), and commercialized by Affymetrix, Inc. of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (International Publication No. WO 99/63385). This approach is generally limited to probes of about 25 nucleotides in length or less. See also Warrington et al., 2000.

Contact Printing. Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8 ×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al., 1994. A recent modification of solid pins involves the use of solid pin tips having concave bottoms, which print more efficiently than flat pins in some circumstances. See Rose 2000.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based et al. tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al., 1995. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al., 2000. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix, Inc. of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized in Rose 2000.

Noncontact Ink-Jet Printing. A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352.

Devices that provide thermal pressure, sonic pressure, or oscillatory pressure on a liquid stream or surface can also be used for ink-jet printing. See Theriault et al., 1999.

Syringe-Solenoid Printing. Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524.

Electronic Addressing. This method involves placing charged molecules at specific positions on a blank microarray substrate, for example a NANOCHIP™ substrate (Nanogen Inc., San Diego, Calif., United States of America). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 and International Publication No. WO 01/23082.

Nanoelectrode Synthesis. An alternative array that can also be used in accordance with the methods of the presently claimed subject matter provides ultra small structures (nanostructures) of a single or a few atomic layers synthesized on a semiconductor surface such as silicon. The nanostructures can be designed to correspond precisely to the three-dimensional shape and electrochemical properties of molecules, and thus can be used to recognize nucleic acids of a particular nucleotide sequence. See U.S. Pat. No. 6,123,819.

V. Hybridization

V.A. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen 1993. In general, a signal to noise ratio of 2-fold (or higher) than that observed for a negative control probe in a same hybridization assay indicates detection of specific or substantial hybridization.

It is understood that in order to determine a gene expression level by hybridization, a full-length cDNA need not be employed. To determine the expression level of a gene represented by one of SEQ ID NOs: 1-70, any representative fragment or subsequence of the sequences set forth in SEQ ID NOs: 1-70 can be employed in conjunction with the hybridization conditions disclosed herein. As a result, a nucleic acid sequence used to assay a gene expression level can comprise sequences corresponding to the open reading frame (or a portion thereof), the 5′ untranslated region, and/or the 3′ untranslated region. It is understood that any nucleic acid sequence that allows the expression level of a reference gene to be specifically determined can be employed with the methods and compositions of the presently claimed subject matter.

V.B. Hybridization on a Solid Support

In another embodiment of the presently claimed subject matter, an amplified and labeled nucleic acid sample is hybridized to probes or probe sets that are immobilized on a continuous solid support comprising a plurality of identifying positions.

Representative hybridization conditions are set forth herein. For some high-density glass-based microarray experiments, hybridization at 65° C. is too stringent for typical use, at least in part because the presence of fluorescent labels destabilizes the nucleic acid duplexes (Randolph & Waggoner 1997). Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 and 6,245,508.

V.C. Hybridization in Solution

In another embodiment of the presently claimed subject matter, an amplified and labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. (See Sambrook & Russell 2001 for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1 M Na+ion, typically about 0.01M to 1M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single probe set is hybridized to an amplified and labeled RNA sample derived from a target nucleic acid sample. Following hybridization, an antibody that recognizes DNA:RNA hybrids is used to precipitate the hybrids for subsequent analysis. The expression level of the gene is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To determine the expression levels of multiple genes simultaneously, probes or probe sets can be distinguished by differential labeling of probes or probe sets. Alternatively, probes or probe sets can be spatially separated in different hybridization vessels. Representative embodiments of each approach are described herein below.

In one embodiment, a probe or probe set having a unique label is prepared for each gene to be analyzed. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech, Piscataway, N.J., United States of America), which can be analyzed with good contrast and minimal signal leakage.

A unique label for each probe or probe set can further comprise a labeled microsphere to which a probe or probe set is attached. A representative system is LabMAP (Luminex Corporation, Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the presently claimed subject matter, an individual probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the amplified, labeled nucleic acid sample with a set of microspheres comprising probe sets, the hybridization mixture is analyzed to detect the signal of the color-code as well as the label of a sample nucleic acid bound to the microsphere. See Vignali 2000; Smith et al., 1998; International Publication Nos. WO 01/13120, WO 01/14589, WO 99/19515, and WO 97/14028.

VI. Detection

Methods for detecting a hybridization duplex or triplex are selected according to the label employed.

In the case of a radioactive label (e.g., ³²P-, ³³P-, or ³⁵S-dNTP) detection can be accomplished by autoradiography or by using a phosphorimager as is known to one of skill in the art. In one embodiment, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al, 1996.

In another embodiment, a nucleic acid sample or probes are labeled with far infrared, near infrared, or infrared fluorescent dyes. Following hybridization, the mixture of amplified nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737; 5,571,388; 5,346,603; 5,534,125; 5,360,523; 5,230,781; 5,207,880; and 4,729,947. An ODYSSEY™ infrared imaging system (Li-Cor, Inc., Lincoln, Nebr., United States of America) can be used for data collection and analysis.

If an epitope label has been used, a protein or compound that binds the epitope can be used to detect the epitope. For example, an enzyme-linked protein can be subsequently detected by development of a calorimetric or luminescent reaction product that is measurable using a spectrophotometer or luminometer, respectively.

In one embodiment, INVADER® technology (Third Wave Technologies, Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717; 5,985,557; 5,994,069; 6,001,567; and 6,090,543.

In another embodiment, target nucleic acid/probe complexes are detected using an amplifying molecule, for example a poly-dA oligonucleotide as described in Lisle et al., 2001. Briefly, a tethered probe is employed against a target nucleic acid having a complementary nucleotide sequence. A target nucleic acid having a poly-dt sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

Surface plasmon resonance spectroscopy can also be used to detect hybridization duplexes formed between a randomly amplified nucleic acid and a probe as disclosed herein. See e.g., Heaton et al., 2001; Nelson et al., 2001; Guedon et al., 2000.

VII. Autoimmune Disease Gene Expression Equation

VII.A. General Description of the Equation

Genes that were the most underexpressed in patients with SLE compared to control population with greatest statistical significance were chosen to determine if they could be used to classify individuals with autoimmune disease and predict whether new samples were derived from autoimmune or control individuals.

TABLE 1
Genes Used in the Equation

| Gene | | SEQ ID |
|---|---|---|
| Symbol | Gene Name | NOs: |
| TGM2 | transglutaminase 2 | 1, 2 |
| SSP29 | silver-stainable protein 29 | 3, 4 |
| TAF2I | TAF11 RNA polymerase II, TATA box | 5, 6 |
| | binding protein-associated factor, 28 | |
| | kilodalton | |
| LLGL2 | lethal giant larvae homolog 2 | 7, 8 |
| TNFAIP2 | tumor necrosis factor, alpha-induced protein | 9, 10 |
| | 2 | |
| SIP1 | survival of motor neuron protein interacting | 11, 12 |
| | protein 1 | |
| BPHL | biphenyl hydrolase-like | 13, 14 |
| TP53 | human tumor protein p53 | 15, 16 |
| DIPA | hepatitis delta antigen-interacting protein A | 17, 18 |
| ASL | argininosuccinate lyase | 19, 20 |
| GNB5 | human guanine nucleotide binding protein, | 21, 22 |
| | beta 5 | |
| MAN1A1 | mannosidase, alpha, class 1A, member 1 | 23, 24 |
| — | EST | 25, 26 |
| LOC51643 | CGI-119 protein | 27, 28 |
| BMP8 | bone morphogenetic protein 8 | 29, 30 |
| — | human mRNA for cytochrome b5, partial | 31, 32 |
| | coding sequence | |
| ORC1L | origin recognition complex, subunit 1-like | 33, 34 |
| — | EST | 35, 36 |
| CDH1 | cadherin 1, type 1, E-cadherin | 37, 38 |
| SUDD | human sudD suppressor of bimD6 homolog | 39, 40 |
| | (SUDD) | |
| EPB72 | erythrocyte membrane protein band 7.2 | 41, 42 |
| CDKN1B | cyclin-dependent kinase inhibitor 1B | 43, 44 |
| CASP6 | caspase 6 | 45, 46 |
| TXK | TXK tyrosine kinase | 47, 48 |
| MYO1C | myosin IC | 49, 50 |
| — | EST | 51, 52 |
| HSJ2 | heat shock protein, DNAJ-like 2 | 53, 54 |
| BRCA1 | breast cancer 1, early onset, transcript | 55, 56 |
| | variant BRCA1a | |
| GUCY1B3 | guanylate cyclase 1, soluble, beta 3 | 57, 58 |
| AP3S2 | adaptor-related protein complex 3, sigma 2 | 59, 60 |
| | subunit | |
| — | EST | 61, 62 |
| SC65 | synaptonemal complex protein 65 | 63, 64 |
| UBE2G2 | ubiquitin-conjugating enzyme E2G 2 | 65, 66 |
| SLC16A4 | solute carrier family 16, member 4 | 67, 68 |
| MMP17 | matrix metalloproteinase 17 | 69, 70 |

VII.B. Use of the Equations to Predict the Presence of Autoimmune Disease

The expression level of each of the genes listed in Table 1 was determined as described hereinabove. For each gene, the average expression level in the control population and the SLE population was summed and divided by 2 (i.e. (controlₐᵥₑ+SLEₐᵥₑ)/2). After determining this value, the expression levels of each of the 35 genes were examined for each subject. For each gene, a value of 0 was assigned for that gene in that subject if the expression level for that gene was less than the average expression level as determined above. If the individual subject's expression level was higher than the average expression level, that gene was assigned a value of 1. The assigned values were then added to arrive at a score (minimum=0; maximum=35).

The range of scores for control individuals was 18-35, and 8 out of 11 control individuals achieved a score of 35. When this analysis was applied to the normal immune subjects, the scores ranged from 26-35. In contrast, however, the range of scores for subjects with autoimmune disease was as follows: 0-5 for SLE; 0-6 for RA; 0-1 for type 1 diabetes; and 0 for MS (p<0.000001).

A group of SLE and RA patients not included in the initial analysis were then tested to examine the predictive value of the above disclosed strategy. The range of scores obtained in these patients was 0-5 for SLE and 0-6 for RA. Thus, the methods disclosed herein can be used to detect the presence or absence of autoimmune disease in a subject whose disease status is unknown by subjecting total RNA isolated from the subject to the aforementioned analysis and generating a score as previously described. In this embodiment, scores of 8 or less suggest the presence of autoimmune disease, while scores of 15 or above suggest the absence of autoimmune disease.

### EXAMPLES

The following Examples have been included to illustrate modes of the presently claimed subject matter. Certain aspects of the following Examples are described in terms of techniques and procedures found or contemplated by the present inventors to work well in the practice of the presently claimed subject matter. These Examples illustrate standard laboratory practices of the inventors. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### Patient Population

Nine control subjects (27-58 years of age) were studied before and after influenza vaccination. Patients with RA (n=20; 46-68 years of age), SLE (n=24; 22-73 years), type 1 diabetes (n=5; 20-46 years), and MS (n =4; 37-54 years) were also enrolled in the study. A clinical diagnosis of each autoimmune disorder was the sole criterion for inclusion. Unaffected family members were also included in the study (n=4, 33-54 years); three were parents of individuals with SLE and one was the child of an individual with RA. The ratio of females to males in the test groups was approximately 3:1.

### Example 2

### Sample Preparation

Peripheral blood mononuclear cells (PBMC) were isolated from heparinized blood drawn from the population of Example 1 by centrifugation on a Ficoll-Hypaque (Sigma-Aldrich, St. Louis, Mo., United States of America) gradient. Leukocyte distribution in PBMC was determined by flow cytometry. Total RNA was isolated with TRI REAGENT® according to the manufacturer's protocol (Molecular Research Center, Cincinnati, Ohio, United States of America).

RNA Labeling. RNA labeling required three steps: priming, elongation, and probe purification. For priming, 1-10 μg of total RNA (in a volume of less than 8.0 μl diethylpyrocarbonate (DEPC)-treated water) and 2.0 μg oligo-dt (10-20 mer mixture; 1 μg/μl) were mixed in a total volume of 10 μl (balance DEPC-treated water) in a 1.5 ml microcentrifuge tube. The tube was placed at 70° C. for 10 minutes and then briefly chilled on ice. For elongation, 6.0 μl 5× First Strand Buffer (Invitrogen catalogue number Y00146), 1.0 μl 0.1 M DTT, 1.5 μl dNTP mixture (each dNTP at 20 mM), and 1.5 μl SUPERSCRIPT™ II reverse transcriptase (Invitrogen) was added to the microcentrifuge tube. 10 μl ³³P-dCTP (10 mCi/ml; specific activity 3000 Ci/mmol; ICN Biomedicals Inc., Irvine, Calif., United States of America) was added to the microcentrifuge tube, the contents mixed thoroughly, and the tube was incubated at 37° C. for 90 minutes. Probe purification was accomplished by passing the elongation reaction mixture through a Bio-Spin 6 chromatography column (Bio-Rad Laboratories, Hercules, Calif., United States of America).

Hybridization of the Labeled RNA to the Membrane. 5 μg of 33P-labeled total RNA isolated from PBMCs were hybridized to GF211 GENEFILTERS® membranes (RESGEN™, a division of Invitrogen Corporation, Carlsbad, Calif., United States of America; the genes present on the GF211 membrane can be found at RESGEN™'s ftp site: ftp://ftp.resgen.com/pub/GENEFILTERS). Prior to hybridization, the filter was pre-treated with 0.5% SDS. The SDS solution was heated to boiling and poured over the membrane, which was then incubated in the SDS solution with gentle agitation for 5 minutes.

After pre-treatment, the filter was prehybridized by placing the filter in a hybridization roller tube (35×150 mm; DNA side facing the interior of the tube) and 5 ml MICROHYB™ solution (RESGEN™) is added to the tube. Additional blocking agents (5 μg COT-1® DNA, Invitrogen Corporation, Carlsbad, Calif., United States of America; 5 μg poly-dA) were added and the tube was vortexed to mix thoroughly. Bubbles between the membrane and the tube were removed and the membranes were incubated in the prehybridization solution at 42° C. for at least 2 hours. For hybridization, the probe was denatured by boiling, cooled, and pipetted into the roller tube containing the GENEFILTERS® membrane and prehybridization solution. The now denatured probe-containing solution was mixed by vortexing. Hybridization occurred overnight, or alternatively for at least 12-18 hours, at 42° C.

Post-Hybridization Washes and Imaging. After hybridization, the filters were washed in the roller tube. The following wash conditions were used: first and second washes were in 2×SSC/1% SDS/50° C. for 20 minutes; third wash was in 0.5×SSC/1% SDS/55° C. for 15 minutes. After washing, the membrane was wrapped in plastic wrap and placed in a phosphorimaging cassette. Filters were exposed to imaging screens for 2-4 hours (short exposure) and then an additional 24 hours (long exposure) and screens were scanned using a PHOSPHORIMAGER™ apparatus (Molecular Dynamics, Piscataway, N.J., United States of America). Data were normalized to yield an average intensity of 1.0 for each clone (4329 clones total) represented on the microarray. Reproducibility of the method was established by performing replicate hybridizations to separate microarrays. Linear regression analysis demonstrated that separate hybridizations yielded R² values ranging from 0.87 to 0.96. Different exposure lengths of identical filters also produced high R² values (0.99).

### Example 3

### Data Analysis

Following phosphorimaging, data were collected in digital format and normalized against a common control filter using the Pathways 3.0 software program (available from Invitrogen). Eisen's Cluster and Treeview software (Stanford University, Palo Alto, Calif., United States of America; (Eisen et al., 1998) were used to compare similarities among individual samples. Data sets were analyzed using hierarchical, K-means, and self-organizing map algorithms (Sherlock 2000). The PATHWAYS™ 3.0 program (RESGEN™) was used to identify differentially expressed genes in the immune and autoimmune disease classes. Expression levels of genes that did not change significantly (99% confidence, Chen test) over any of the conditions were removed from the database (Kim et al., 2000). The remaining genes in the data set were clustered using an unsupervised K-means clustering algorithm with ten centroids (Eisen et al., 1998; Sherlock 2000).

### Example 4

### Gene Expression Profiles During a Normal Immune Response

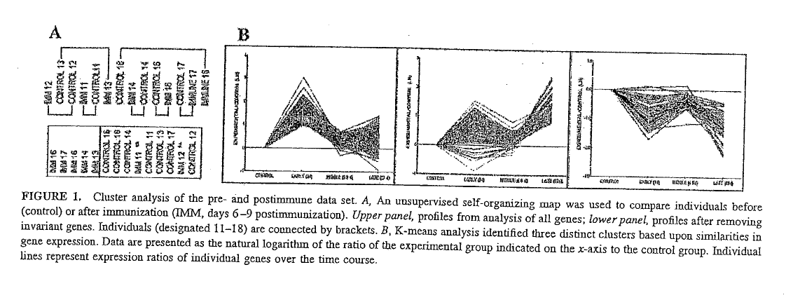

To test the hypothesis that the mononuclear cell population represented a suitable source to measure alterations in gene expression, changes in gene expression in PBMC from healthy control subjects (n=9) were measured before and after immunization with influenza vaccine. It was most likely that a gene expression profile derived from these subjects would involve a secondary immune response because all subjects had prior exposure to many influenza antigens (Ags). Samples were collected from subjects at three time points: 3, 6-9, and 19-21 days after immunization. A self-organizing map algorithm was used to compare the preimmune to the immune group. This method segregated individuals based upon identity rather than immune status, as demonstrated by the relative proximity of individual samples (See FIG. 1A, upper panel). Thus, total gene expression patterns remained relatively unchanged after immunization. To focus on distinctions that arose from the most differentially expressed genes, genes for which expression levels did not vary by more than 3 standard deviations (SD) from their respective means were filtered out. After filtering, expression profiles were segregated primarily by pre- and postimmune status (See FIG. 1A, lower panel), suggesting that uniform changes in expression levels of a smaller subset of genes distinguished pre- and postimmunization groups. To identify these genes, K-means clustering was used to group genes on the basis of similarity in expression patterns.

Three distinct clusters associated with the normal immune response were found (See FIG. 1B). The first cluster consisted of 304 genes that were overexpressed 3 days after immunization. This cluster mainly contained genes that encode proteins involved in key signal transduction pathways (e.g., protein kinase C, phospholipase C, 1,2-diacylglycerol kinase, mitogen-activated protein kinase, STATs and STAT inhibitors, AP-1 transcription factors, interferon regulatory factors, and proteins required for proliferation). Genes in this cluster exhibited an increase in expression from 3- to 21-fold compared with the control group.

The second cluster of 88 late (19-21 days) response genes represented a shift away from signaling and proliferation pathways toward increased functional activity. Among the late immune response gene cluster, chemokines (SCYA3, SCYA13, SCYA14), complement components (CIS), interferon (IFN) -inducible proteins (IFI35), and leukocyte homing/adhesion (ICAM2) genes were overexpressed. Receptors for serotonin, glutamate, estrogen, and retinoic acid were also overexpressed. Increases in expression levels of this group of genes varied from 2- to 11 -fold.

The final immune response cluster contained 78 genes that exhibited reduced expression levels over the entire time course. Over 15% of these genes encode ribosomal proteins. This represents a decrease in the expression of one-third of all ribosomal protein encoding genes present on the microarrays. Coordinate changes in ribosomal protein gene expression have been linked to differentiation in eukaryotic cells (Krichevsky et al., 1999) and the observed changes could reflect differentiation of lymphocytes to an effector state in response to immunization. While applicants do not wish to be bound by any particular theory of operation, taken together, these data illustrate dynamic, coordinate changes in mRNA expression that accompany the immune response in vivo. First, genes appeared to be induced that are required for signal transduction and cell proliferation, two key elements of the early immune response. Later, a shift away from these genes to other classes that are necessary to undertake the immune functions of lymphocytes occurred.

### Example 5

### Expression Profiles of Immunized Subiects Versus Autoimmune Patients

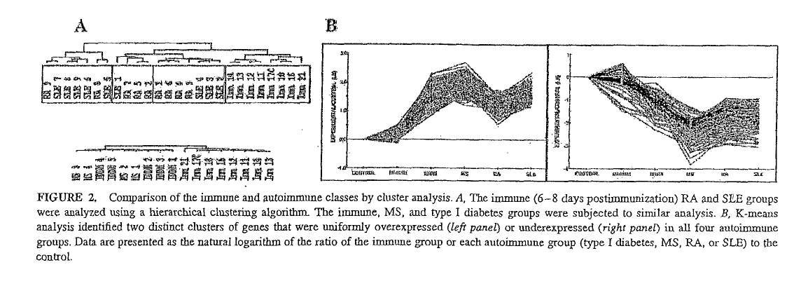

In order to determine if the observations described above are differ between subjects undergoing a normal immune response (i.e. subjects immunized with influenza vaccine) and subjects undergoing an autoimmune response, samples were obtained from patients diagnosed with one of four common autoimmune disorders: RA, MS, type 1 diabetes, and SLE. The relatedness of global gene expression profiles associated with autoimmune disease was examined relative to the normal immune response using a hierarchical clustering algorithm (See FIG. 2A). Other clustering algorithms yielded similar results. Comparison between the RA/SLE class and the normal immune response class yielded four major branches from the clustering analysis. One major branch contained all normal immune samples and none of the autoimmune samples. The autoimmune samples segregated into the other three major branches. This analysis revealed that some of the RA samples (e.g., RA2 and RA5, or RA1, RA6, and RA4) and some of the SLE samples (e.g., SLE2, SLE3, and SLE4, or SLE6, SLE8, and SLE9) were highly related. However, unlike distinctions between the RA/SLE and the normal immune response samples, it was not possible to segregate the majority of RA samples from the majority of SLE samples, suggesting that RA and SLE might represent a common autoimmune class that is distinct from the immune class. Similar results were obtained from clustering of normal immune response samples with MS/type 1 diabetes samples. Again, there was good segregation of the normal immune response group from the MS/type 1 diabetes group, but MS and type 1 diabetes profiles did not segregate from each other. This inability to segregate within autoimmune class was retained even when invariant genes were removed from the data set.

The data set was further analyzed to identify genes that were most differentially expressed in autoimmune diseases relative to the normal immune response. Non-autoimmune groups were segregated into control (no treatment) and immune (6-9 days after immunization). Individual samples from the autoimmune groups were segregated based upon disease type and compared with the immune response gene profiles. Gene expression differences among different groups were plotted as the natural logarithm of the ratio between experimental condition and control group.

Two clusters of differentially expressed genes distinguished between (1) patients with autoimmune disease, and (2) control and immune individuals (See FIG. 2B). The first major cluster comprised 95 genes that were overexpressed in all four autoimmune diseases (type 1 diabetes, MS, RA, and SLE). The genes in this overexpressed autoimmune cluster were relatively heterogeneous, representing several distinct functional categories: receptors (CSF3R, HLA-DMB, HLALS, TGFBR2, and BMPR2), inflammatory mediators (MSTP9, BDNF, CES1, ELA3, and CYR61), signaling/second messenger molecules (FASTK, DGKA, and DGKD), and autoantigens (GARS and GAD2). The second major cluster contained 117 genes that were strongly underexpressed in all autoimmune groups. Levels of expression of these genes did not change in the immune response group. Many of the down-regulated genes play key roles in apoptosis (TRADD, TRAP1, TRIP, TRAF2, CASP6, CASP8, TP53, and SIVA) and ubiquitin/proteasome function (UBE2M, UBE2G2, and POH1). Inhibitors of various cellular functions were also widely represented in this cluster. These include direct inhibitors of cell cycle progression (CDKN1B, CDKN2A, and BRCA1), as well as inducers of cell differentiation (LIF and CD24). Certain enzyme inhibitors (APOC3 and KALL) were also found in this class.

K-means clustering indicated that it was not possible to identify clusters of genes that overlapped between the immune and autoimmune classes, suggesting that the gene expression patterns that characterize the normal immune response are considerably different from those found in autoimmune disease. In addition, clusters of genes that distinguished among the distinct autoimmune diseases were not found, suggesting that the autoimmune diseases studied are more similar to each other than they are to a normal immune response.

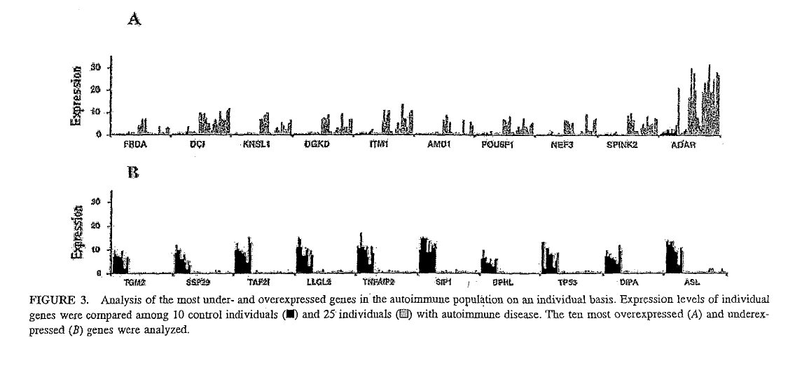

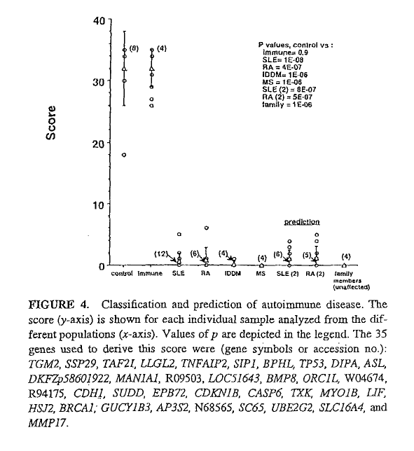

The expression levels of single genes between preimmune controls and individuals with each of four autoimmune diseases were investigated further. Ten genes were chosen that exhibited the greatest level of over- and underexpression (see FIGS. 3A and 3B) at the population level and were highly consistent in each individual with autoimmune disease. Overexpressed genes in the autoimmune population showed greater individual variation (see FIG. 3A). Among the overexpressed genes, no individual gene was overexpressed in all autoimmune individuals compared with all control individuals. However, each of these overexpressed genes was significantly overexpressed in the autoimmune population considered together when compared to the control population taken as a whole (p<0.05). In contrast, the expression levels of the underexpressed genes (FIG. 3B) were lower in each autoimmune individual than in any control individual.

Differences in gene expression between the control and the autoimmune populations might be attributed to alterations in distribution or activation status of cells that make up the PBMC. Two analyses were performed to test this possibility. First, PBMC preparations were analyzed for frequency of CD3 (T cells), CD14 (monocytes), CD19 (B cells), and leukocyte alkaline phosphatase (neutrophils) by flow cytometry. All PBMC preparations from both subject groups contained 75-80% T cells, about 10% monocytes, about 5% B cells, and less than 1% neutrophils. Second, it was determined whether expression levels of genes that are either restricted to a given subpopulation or reflect activation status were differentially expressed in the control compared with the autoimmune population (Table 2). Expression levels of these genes varied by less than 2-fold between the control and autoimmune groups and this difference did not achieve statistical significance. Taken together, these data suggest that alterations in the composition or activation status of PBMC did not account for the observed differences in gene expression between the control and autoimmune populations.

TABLE 2
Expression Levels of Genes Encoding Proteins that Distinguish
Among Lymphocyte Subsets or Activation State

| | Control | SLE | RA | IDDM | MS |
|---|---|---|---|---|---|
| T cell Ags | | | | | |
| CD3δ | 0.7 ± 0.2^{a} | 0.6 ± 0.4 | 0.5 ± 0.2 | 0.5 ± 0.2 | 0.4 ± 0.2 |
| CD3γ | 0.5 ± 0.1 | 0.6 ± 0.9 | 0.4 ± 0.1 | 0.3 ± 0.1 | 0.4 ± 0.1 |
| CD8β (Tc) | 0.8 ± 0.3 | 0.8 ± 0.2 | 0.6 ± 0.2 | 0.5 ± 0.2 | 0.5 ± 0.2 |
| CD44 | 0.5 ± 0.1 | 0.8 ± 0.5 | 0.7 ± 0.4 | 0.8 ± 0.5 | 0.7 ± 0.4 |
| (memory) | | | | | |
| CD69 | 0.5 ± 0.2 | 0.7 ± 0.3 | 0.6 ± 0.2 | 0.8 ± 0.3 | 0.7 ± 0.4 |
| (activation) | | | | | |
| CD62 | 1.3 ± 0.6 | 1.4 ± 0.9 | 1.8 ± 0.1 | 1.7 ± 1.1 | 1.9 ± 1.1 |
| (L-selectin) | | | | | |
| CD122 | 0.4 ± 0.1 | 0.4 ± 0.2 | 0.5 ± 0.2 | 0.3 ± 0.1 | 0.3 ± 0.1 |
| (IL-2R β) | | | | | |
| B Cell Ags | | | | | |
| CD79a | 0.6 ± 0.3 | 0.4 ± 0.2 | 0.4 ± 0.2 | 0.4 ± 0.2 | 0.4 ± 0.2 |
| CD79b | 0.5 ± 0.2 | 0.6 ± 0.3 | 0.8 ± 0.7 | 0.8 ± 0.4 | 0.7 ± 0.3 |
| CD72 | 0.4 ± 0.1 | 0.4 ± 0.3 | 0.4 ± 0.2 | 0.3 ± 0.1 | 0.3 ± 0.1 |
| CD22 | 0.3 ± 0.1 | 0.4 ± 0.3 | 0.4 ± 0.4 | 0.3 ± 0.1 | 0.3 ± 0.1 |

Monocyte Ags

| | | | | | |
|---|---|---|---|---|---|
| CD14 | 0.5 ± 0.2 | 0.4 ± 0.2 | 0.3 ± 0.1 | 0.3 ± 0.2 | 0.3 ± 0.2 |
| CD163 | 0.3 ± 0.1 | 0.4 ± 0.2 | 0.4 ± 0.2 | 0.3 ± 0.1 | 0.3 ± 0.2 |
| CD32 | 0.3 ± 0.1 | 0.5 ± 0.4 | 0.5 ± 0.3 | 0.3 ± 0.1 | 0.4 ± 0.2 |
| (B/mθ) | | | | | |

Activation-induced Ags

| | | | | | |
|---|---|---|---|---|---|
| CD54 | 4.4 ± 1.8 | 3.1 ± 2.1 | 4.3 ± 0.7 | 4.3 ± 2.2 | 3.9 ± 1.0 |
| (ICAM-1) | | | | | |
| CD38 | 0.4 ± 0.3 | 0.3 ± 0.2 | 0.3 ± 0.1 | 0.3 ± 0.1 | 0.3 ± 0.1 |
| CD71 | 0.2 ± 0.1 | 0.2 ± 0.2 | 0.2 ± 0.1 | 0.2 ± 0.1 | 0.2 ± 0.1 |

^{a}Average Expression Level ± SD

### Example 6

### Fluorescent Labeling of Nucleic Acids

A nucleic acid sample can be used as a template for direct incorporation of fluorescent nucleotide analogs (e.g., Cy3-dUTP and Cy5- dUTP, available from Amersham Pharmacia Biotech of Piscataway, N.J., United States of America) by a polymerization reaction. In brief, a 50 μl labeling reaction can contain 2 μg of template DNA, 5 μl of 10×buffer, 1.5 μl of fluorescent dUTP, 0.5 μl each of dATP, dCTP, and dGTP, 1 μl of hexamers and decamers (i.e. primers, whether random or derived from a gene of interest), and 2 μl of Klenow (E. coli DNA polymerase 3′ to 5′ exo- from New England Biolabs of Beverly, Mass., United States of America).

### Example 7

### Noncovalent Binding of Nucleic Acid Probes onto Glass

PCR fragments are suspended in a solution of 3 to 5M NaSCN and spotted onto amino-silanized slides using a GMS 417™ arrayer from Affymetrix of Santa Clara, Calif., United States of America. After spotting, the slides are heated at 80° C. for 2 hours to dehydrate the spots. Prior to hybridization, the slides are washed in isopropanol for 10 minutes, followed by washing in boiling water for 5 minutes. The washing steps remove any nucleic acid that is not bound tightly to the glass and help to reduce background created by redistribution of loosely attached DNA during hybridization. Contaminants such as detergents and carbohydrates should be minimized in the spotting solution. See also Maitra & Thakur 1992; Maitra & Thakur 1994.

### Example 8

### Hybridization to a Microarray Comprising Gene-specific Probes

Labeled nucleic acids from the sample are prepared in a solution of 4×SSC buffer, 0.7 μg/μl tRNA, and 0.3% SDS to a total volume of 14.75 μl. The hybridization mixture is denatured at 98° C. for 2 minutes, cooled to 65° C., applied to the microarray, and covered with a 22-mm² cover slip. The slide is placed in a waterproof hybridization chamber for hybridization in a 65° C. water bath for 3 hours. Following hybridization, slides are washed in 1×SSC buffer with 0.06% SDS followed by 2 minutes in 0.06×SSC buffer.

### REFERENCES

The references listed below as well as all references cited in the specification are incorporated herein by reference to the extent that they supplement, explain, provide a background for, or teach methodology, techniques, and/or compositions employed herein.

Albert J, Wahlberg J, Lundeberg J, Cox S, Sandstrom E, Wahren B & Uhlen M (1992) Persistence of Azidothymidine-Resistant Human Immunodeficiency Virus Type 1 RNA Genotypes in Posttreatment Sera. J Virol 66:5627-5630.

Alexay C, Kain R C, Hanzel D K & Johnston R F (1996) Fluorescence scanner employing a macro scanning objective, in Menzel E R, ed, Fluorescence Detection IV. Proc SPIE 2705:63-72.

Altschul S F, Gish W, Miller W, Myers E W & Lipman D J (1990) Basic Local Alignment Search Tool. J Mol Biol 215:403-410.

Ausubel F M, Brent R, Kingston R E, Moore D D, Seidman J G, Smith J A & Struhl K, eds (1994) Current Protocols in Molecular Biology. Wiley, New York.

Bej A K, Mahbubani M H, Dicesare J L & Atlas R M (1991) Polymerase Chain Reaction-Gene Probe Detection of Microorganisms by Using Filter-Concentrated Samples. Appl Environ Microbiol 57:3529-3534.

Boom R, Sol C J, Salimans M M, Jansen C L, Wertheim-van Dillen P M & van der Noordaa J (1990) Rapid and Simple Method for Purification of Nucleic Acids. J Clin Microbiol 28:495-503.

Buffone G J, Demmler G J, Schimbor C M & Greer J (1991) Improved Amplification of Cytomegalovirus DNA from Urine after Purification of DNA with Glass Beads. Clin Chem 37:1945-1949.

Busch M P, Wilber J C, Johnson P, Tobler L & Evans C S (1992) Impact of Specimen Handling and Storage on Detection of Hepatitis C Virus RNA. Transfusion 32:420-425.

Cha R S & Thilly W G (1993) Specificity, Efficiency, and Fidelity of Pcr. PCR Methods Appl 3:S18-29.

Chiodi F, Keys B, Albert J, Hagberg L, Lundeberg J, Uhlen M, Fenyo E M & Norkrans G (1992) Human Immunodeficiency Virus Type 1 Is Present in the Cerebrospinal Fluid of a Majority of Infected Individuals. J Clin Microbiol 30:1768-1771.

DeRisi J, Penland L, Brown P O, Bittner M L, Meltzer P S, Ray M, Chen Y, Su Y A & Trent J M (1996) Use of a cDNA Microarray to Analyse Gene Expression Patterns in Human Cancer. Nat Genet 14:457-460.

Dubiley S, Kirillov E, Lysov Y & Mirzabekov A (1997) Fractionation, Phosphorylation and Ligation on Oligonucleotide Microchips to Enhance Sequencing by Hybridization. Nucleic Acids Res 25:2259-2265.

Eberwine J, Yeh H, Miyashiro K, Cao Y, Nair S, Finnell R, Zettel M & Coleman P (1992) Analysis of Gene Expression in Single Live Neurons. Proc Natl Acad Sci U S A 89:3010-3014.

Eisen M B, Spellman P T, Brown P O & Botstein D (1998) Cluster Analysis and Display of Genome-Wide Expression Patterns. Proc Natl Acad Sci U S A 95:14863-14868.

Englert D (2000) in Schena M, ed, Microarray Biochip Technology, pp. 231-246, Eaton Publishing, Natick, Mass., United States of America.

Fodor S P, Read J L, Pirrung M C, Stryer L, Lu A T & Solas D (1991) Light-Directed, Spatially Addressable Parallel Chemical Synthesis. Science 251:767-773.

Fodor S P, Rava R P, Huang X C, Pease A C, Holmes C P & Adams C L (1993) Multiplexed Biochemical Assays with Biological Chips. Nature 364:555-556.

Guedon P, Livache T, Martin F, Lesbre F, Roget A, Bidan G & Levy Y (2000) Characterization and Optimization of a Real-Time, Parallel, Label-Free, Polypyrrole-Based DNA Sensor by Surface Plasmon Resonance Imaging. Anal Chem 72:6003-6009.

Hamel A L, Wasylyshen M D & Nayar G P (1995) Rapid Detection of Bovine Viral Diarrhea Virus by Using RNA Extracted Directly from Assorted Specimens and a One-Tube Reverse Transcription Pcr Assay. J Clin Microbiol 33:287-291.

Heaton R J, Peterson A W & Georgiadis R M (2001) Electrostatic Surface Plasmon Resonance: Direct Electric Field-Induced Hybridization and Denaturation in Monolayer Nucleic Acid Films and Label-Free Discrimination of Base Mismatches. Proc Natl Acad Sci U S A 98:3701-3704.

Henikoff S & Henikoff J G (1992) Amino Acid Substitution Matrices from Protein Blocks. Proc Nat Acad Sci U S A 89:10915-10919.

Hermanson G T (1990) Bioconjugate Techniques, Academic Press, San Diego, Calif., United States of America.

Herrewegh A A, de Groot R J, Cepica A, Egberink H F, Horzinek M C & Rottier P J (1995) Detection of Feline Coronavirus RNA in Feces, Tissues, and Body Fluids of Naturally Infected Cats by Reverse Transcriptase Pcr. J Clin Microbiol 33:684-689.

Izraeli S, Pfleiderer C & Lion T (1991) Detection of Gene Expression by Pcr Amplification of RNA Derived from Frozen Heparinized Whole Blood. Nucleic Acids Res 19:6051.

Jacobson D L, Gange S J, Rose N R & Graham N M (1997) Epidemiology and Estimated Population Burden of Selected Autoimmune Diseases in the United States. Clin Immunol Immunopathol 84:223-243.

Joyce C (2002) Quantitative RT-PCR. A Review of Current Methodologies. Methods Mol Biol 193:83-92.

Karlin S & Altschul S F (1993) Applications and Statistics for Multiple High-Scoring Segments in Molecular Sequences. Proc Natl Acad Sci U S A 90:5873-5877.

Kim S, Dougherty E R, Chen Y, Sivakumar K, Meltzer P, Trent J M & Bittner M (2000) Multivariate Measurement of Gene Expression Relationships. Genomics 67:201-209.

Kohsaka H & Carson D A (1994) Solid-Phase Polymerase Chain Reaction. J Clin Lab Anal 8:452-455.

Kotzin B L (1996) Systemic Lupus Erythematosus. Cell 85:303-306.

Krichevsky A M, Metzer E & Rosen H (1999) Translational Control of Specific Genes During Differentiation of HI-60 Cells. J Biol Chem 274:14295-14305.

Kukreja A & Maclaren N K (2000) Current Cases in Which Epitope Mimicry Is Considered as a Component Cause of Autoimmune Disease: Immune-Mediated (Type 1) Diabetes. Cell Mol Life Sci 57:534-541.

Lanciotti R S, Calisher C H, Gubler D J, Chang G J & Vorndam A V (1992) Rapid Detection and Typing of Dengue Viruses from Clinical Samples by Using Reverse Transcriptase-Polymerase Chain Reaction. J Clin Microbiol 30:545-551.

Linz U, Delling U & Rubsamen-Waigmann H (1990) Systematic Studies on Parameters Influencing the Performance of the Polymerase Chain Reaction. J Clin Chem Clin Biochem 28:5-13.

Lisle C M, Bortolin S, Benight A S, Janeczko R A & Zastawny R L (2001) Novel Signal Amplification Technology with Applications in DNA and Protein Detection Systems. Biotechniques 30:1268-1272.

Liu J & Hlady V (1996) Chemical pattern on silica surface prepared by UV irradiation of 3-mercapto - propyltriethoxy silane layer: Surface characterization and fibrinogen adsorption. Colloids and Surfaces B. Biointerfaces 8:25-37.

Mace M L, Jr., Montagu J, Rose S D & McGuinness G (2000) in Schena M ed, Microarray Biochip Technology, pp. 39-64, Eaton Publishing, Natick, Mass., United States of America

Maier E, Meier-Ewert S, Ahmadi A R, Curtis J & Lehrach H (1994) Application of Robotic Technology to Automated Sequence Fingerprint Analysis by Oligonucleotide Hybridisation. J Biotechnol 35:191-203.

Maitra R & Thakur A R (1992) Curr Sci 62:586-588.

Maitra R & Thakur A R (1994) Multiple Fragment Ligation on Glass Surface: A Novel Approach. Indian J Biochem Biophys 31:97-99.

Marrack P, Kappler J & Kotzin B L (2001) Autoimmune Disease: Why and Where It Occurs. Nat Med 7:899-905.

Martin A, Barbesino G & Davies T F (1999) T-Cell Receptors and Autoimmune Thyroid Disease—Signposts for T-Cell-Antigen Driven Diseases. Int Rev Immunol 18:111-140.

McCaustland K A, Bi S, Purdy M A & Bradley D W (1991) Application of Two RNA Extraction Methods Prior to Amplification of Hepatitis E Virus Nucleic Acid by the Polymerase Chain Reaction. J Virol Methods 35:331-342.

McPherson M J, Hames B D & Taylor G, eds, (1995) PCR 2: A Practical Approach, IRL Press, New York, N.Y., United States of America.

Millar D S, Withey S J, Tizard M L, Ford J G & Hermon-Taylor J (1995) Solid-Phase Hybridization Capture of Low-Abundance Target DNA Sequences: Application to the Polymerase Chain Reaction Detection of Mycobacterium Paratuberculosis and Mycobacterium Avium Subsp. Silvaticum. Anal Biochem 226:325-330.

Natarajan V, Plishka R J, Scott E W, Lane H C & Salzman N P (1994) An Internally Controlled Virion Pcr for the Measurement of Hiv-1 RNA in Plasma. PCR Methods Appl 3:346-350.

Needleman S B & Wunsch C D (1970) A General Method Applicable to the Search for Similarities in the Amino Acid Sequence of Two Proteins. J Mol Biol 48:443-453.

Nelson B P, Grimsrud T E, Liles M R, Goodman R M & Corn R M (2001) Surface Plasmon Resonance Imaging Measurements of DNA and RNA Hybridization Adsorption onto DNA Microarrays. Anal Chem 73:1-7.

O'Donnell M J, Tang K, Koster H, Smith C L & Cantor C R (1997) High-Density, Covalent Attachment of DNA to Silicon Wafers for Analysis by MALDI-TOF Mass Spectrometry. Anal Chem 69:2438-2443.

Paladichuk A (1999) Isolating RNA: Pure and Simple. The Scientist 13(16):20-23.

PCT International Publication No. WO 97/14028.

PCT International Publication No. WO 99/19515

PCT International Publication No. WO 99/63385

PCT International Publication No. WO 01/13120

PCT International Publication No. WO 01/14589

PCT International Publication No. WO 01/23082

Pearson W R & Lipman D J (1988) Improved Tools for Biological Sequence Comparison. Proc Natl Acad Sci U S A 85:2444-2448.

Pietu G, Alibert O, Guichard V, Lamy B, Bois F, Leroy E, Mariage-Sampson R, Houlgatte R, Soularue P & Auffray C (1996) Novel Gene Transcripts Preferentially Expressed in Human Muscles Revealed by Quantitative Hybridization of a High Density Cdna Array. Genome Res 6:492-503.

Quayle A J, Wilson K B, Li S G, Kjeldsen-Kragh J, Oftung F, Shinnick T, Sioud M, Forre O, Capra J D & Natvig J B (1992) Peptide Recognition, T Cell Receptor Usage and HIa Restriction Elements of Human Heat-Shock Protein (Hsp) 60 and Mycobacterial 65-Kda Hsp-Reactive T Cell Clones from Rheumatoid Synovial Fluid. Eur J Immunol 22:1315-1322.

Randolph J B & Waggoner A S (1997) Stability, Specificity and Fluorescence Brightness of Multiply-Labeled Fluorescent DNA Probes. Nucleic Acids Res 25:2923-2929.

Ratner B D & Castner D G (1997) in Vickerman J C, ed, Surface Analysis: The Principal Techniques, John Wiley & Sons, New York, N.Y., United States of America.

Robertson J M & Walsh-Weller J (1998) An Introduction to Pcr Primer Design and Optimization of Amplification Reactions. Methods Mol Biol 98:121-154.

Rose D (2000) in Schena M ed, Microarray Biochip Technology, pp. 19-38, Eaton Publishing, Natick, Mass., United States of America.

Roux K H (1995) Optimization and Troubleshooting in Pcr. PCR Methods Appl 4:S185-194.

Rupp G M & Locker J (1988) Purification and Analysis of RNA from Paraffin-Embedded Tissues. Biotechniques 6:56-60.

Sambrook & Russell (2001) Molecular Cloning: A Laboratory Manual, 3^{rd} Edition, Cold Spring Harbor Laboratory Press, Cold Spring Harbor, N.Y., United States of America.

Sapolsky R J & Lipshutz R J (1996) Mapping Genomic Library Clones Using Oligonucleotide Arrays. Genomics 33:445-456.

Schena M, Shalon D, Davis R W & Brown P O (1995) Quantitative Monitoring of Gene Expression Patterns with a Complementary DNA Microarray. Science 270:467-470.

Schena M, Shalon D, Heller R, Chai A, Brown P O & Davis R W (1996) Parallel Human Genome Analysis: Microarray-Based Expression Monitoring of 1000 Genes. Proc Natl Acad Sci U S A 93:10614-10619.

Shalon D, Smith S J & Brown P O (1996) A DNA Microarray System for Analyzing Complex DNA Samples Using Two-Color Fluorescent Probe Hybridization. Genome Res 6:639-645.

Sherlock G (2000) Analysis of Large-Scale Gene Expression Data. Curr Opin Immunol 12:201-205.

Shoemaker D D, Lashkari D A, Morris D, Mittmann M & Davis R W (1996) Quantitative Phenotypic Analysis of Yeast Deletion Mutants Using a Highly Parallel Molecular Bar-Coding Strategy. Nat Genet 14:450-456.

Shriver-Lake L C (1998) in Cass T & Ligler F S, eds, Immobilized Biomolecules in Analysis, pp. 1-14, Oxford Press, Oxford, United Kingdom.

Smith P L, WalkerPeach C R, Fulton R J & DuBois D B (1998) A Rapid, Sensitive, Multiplexed Assay for Detection of Viral Nucleic Acids Using the Flowmetrix System. Clin Chem 44:2054-2056.

Smith T F & Waterman M (1981) Comparison of Biosequences. Adv Appl Math 2:482-489.

Southern E M (1975) Detection of Specific Sequences among DNA Fragments Separated by Gel Electrophoresis. J Mol Biol 98:503-517.

Steel A, Torres M, Hartwell J, Yu Y Y, Ting N, Hoke G & Yang, H (2000) in Schena M, ed, Microarray Biochip Technology, pp. 87-118, Eaton Publishing, Natick, Mass., United States of America.

Strain S R & Chmielewski J G (2001) ROCK: A Spreadsheet-Based Program for the Generation and Analysis of Random Oligonucleotide Primers used in PCR. BioTechniques 30:1286-1293.

Tanaka S, Minagawa H, Toh Y, Liu Y & Mori R (1994) Analysis by RNA-Pcr of Latency and Reactivation of Herpes Simplex Virus in Multiple Neuronal Tissues. J Gen Virol75 ( Pt 10):2691-2698.

Telenius H, Carter N P, Bebb C E, Nordenskjold M, Ponder B A & Tunnacliffe A (1992) Degenerate Oligonucleotide-Primed Pcr: General Amplification of Target DNA by a Single Degenerate Primer. Genomics 13:718-725.

Theriault T P, Winder S C & Gamble R C (1999) in Schena M, ed, DNA Microarrays: A Practical Approach, pp. 101-120, Oxford University Press Inc., New York, N.Y., United States of America.

Tijssen P (1993) Laboratory Techniques in Biochemistry and Molecular Biology-Hybridization with Nucleic Acid Probes. Elsevier, N.Y.

Ufret-Vincenty R L, Quigley L, Tresser N, Pak S H, Gado A, Hausmann S, Wucherpfennig K W & Brocke S (1998) In Vivo Survival of Viral Antigen-Specific T Cells That Induce Experimental Autoimmune Encephalomyelitis. J Exp Med 188:1725-1738.

U.S. Pat. No. 4,729,947

U.S. Pat. No. 5,346,603

U.S. Pat. No. 5,445,934

U.S. Pat. No. 5,207,880

U.S. Pat. No. 5,230,781

U.S. Pat. No. 5,360,523

U.S. Pat. No. 5,534,125

U.S. Pat. No. 5,571,388

U.S. Pat. No. 5,743,960

U.S. Pat. No. 5,843,767

U.S. Pat. No. 5,846,717

U.S. Pat. No. 5,916,524

U.S. Pat. No. 5,965,352

U.S. Pat. No. 5,985,557

U.S. Pat. No. 5,994,069

U.S. Pat. No. 6,001,567

U.S. Pat. No. 6,066,457

U.S. Pat. No. 6,090,543

U.S. Pat. No. 6,017,696

U.S. Pat. No. 6,086,737

U.S. Pat. No. 6,123,819

U.S. Pat. No. 6,162,603

U.S. Pat. No. 6,225,059

U.S. Pat. No. 6,245,508

Vandesompele J, De Preter K, Pattyn F, Poppe B, Van Roy N, De Paepe A & Speleman F (2002) Acurate Normalization of Real-Time Quantitative RT-PCR Data by Geometric Averaging of Multiple Internal Control Genes. Genome Biol 3:1-12.

Van Gelder R N, von Zastrow M E, Yool A, Dement W C, Barchas J D & Eberwine J H (1990) Amplified RNA Synthesized from Limited Quantities of Heterogeneous cDNA. Proc Natl Acad Sci U S A 87:1663-1667.

Van Kerckhoven I, Fransen K, Peeters M, De Beenhouwer H, Piot P & van der Groen G (1994) Quantification of Human Immunodeficiency Virus in Plasma by RNA Pcr, Viral Culture, and P24 Antigen Detection. J Clin Microbiol 32:1669-1673.

Vignali D A (2000) Multiplexed Particle-Based Flow Cytometric Assays. J Immunol Methods 243:243-255.

Wang A M, Doyle M V & Mark D F (1989) Quantitation of Mrna by the Polymerase Chain Reaction. Proc Nat Acad Sci U S A 86:9717-9721.

Wang E, Miller L D, Ohnmacht G A, Liu E T & Marincola F M (2000) High-Fidelity Mrna Amplification for Gene Profiling. Nat Biotechnol 18:457-459.

Warrington J A, Dee S & Trulson M (2000) in Schena M, ed, Microarray Biochip Technology, pp. 119-148, Eaton Publishing, Natick, Mass., United States of America.

Williams J F (1989) Optimization Strategies for the Polymerase Chain Reaction. Biotechniques 7:762-769.

Williams J G, Kubelik A R, Livak K J, Rafalski J A & Tingey S V (1990) DNA Polymorphisms Amplified by Arbitrary Primers Are Useful as Genetic Markers. Nucleic Acids Res 18:6531-6535.

Worley J et al. (2000) in Schena M, ed, Microarray Biochip Technology, pp. 65-86, Eaton Publishing, Natick, Mass., United States of America,

Yang P, Deng T, Zhao D, Feng P, Pine D, Chmelka B F, Whitesides G M & Stucky G D (1998) Hierarchically Ordered Oxides. Science 282:2244-2246.

Yershov G, Barsky V, Belgovskiy A, Kirillov E, Kreindlin E, lvanov I, Parinov S, Guschin D, Drobishev A, Dubiley S & Mirzabekov A (1996) DNA Analysis and Diagnostics on Oligonucleotide Microchips. Proc Natl Acad Sci U S A 93:4913-4918.

It will be understood that various details of the presently claimed subject matter can be changed without departing from the scope of the presently claimed subject matter. Furthermore, the foregoing description is for the purpose of illustration only, and not for the purpose of limitation.

What is claimed is:

1. A method for detecting an autoimmune disorder in a subject, the method comprising:
(a) obtaining a biological sample from the subject;
(b) determining expression levels of at least two genes in the biological sample; and
(c) comparing the expression level of each gene determined in step (b) with a standard, wherein the comparing detects the presence of an autoimmune disorder in the subject.

2. The method of claim 1, wherein the autoimmune disorder is selected from the group consisting of rheumatoid arthritis (RA), systemic lupus erythematosus (SLE), multiple sclerosis (MS), type 1 (i.e. insulin- dependent) diabetes (IDDM), and combinations thereof.

3. The method of claim 1, wherein the biological sample is a cell.

4. The method of claim 3, wherein the cell is a peripheral blood mononuclear cell.

5. The method of claim 1, wherein the subject is an animal.

6. The method of claim 5, wherein the animal is a mammal.

7. The method of claim 6, wherein the mammal is a human.

8. The method of claim 1, wherein the determining comprises a technique selected from the group consisting of a Northern blot, hybridization to a nucleic acid microarray, and a reverse transcription-polymerase chain reaction (RT-PCR).

9. The method of claim 8, wherein the RT-PCR is quantitative RT-PCR.

10. The method of claim 1, wherein the determining is of the expression levels of at least two genes represented by SEQ ID NOs: 1-70.

11. The method of claim 10, wherein the determining is of the expression levels of at least five genes represented by SEQ ID NOs: 1-70.

12. The method of claim 10, wherein the determining is of the expression levels of at least ten genes represented by SEQ ID NOs: 1-70.

13. The method of claim 10, wherein the determining is of the expression levels of at least twenty genes represented by SEQ ID NOs: 1-70.

14. The method of claim 10, wherein the determining is of the expression levels of at least twenty-five genes represented by SEQ ID NOs: 1-70.

15. The method of claim 10, wherein the determining is of the expression levels of all of the genes represented by SEQ ID NOs: 1-70.

16. The method of claim 1, wherein the comparing comprises:
(a) establishing an average expression level for each gene in a population, wherein the population comprises statistically significant numbers of normal subjects and subjects that have one or more different autoimmune disorders;
(b) assigning a first value to each gene for which the expression level in the subject is higher than the average expression level in the population and a second value to each gene for which the expression level in the subject is lower than the average expression level in the population; and
(c) adding the values assigned in step (b) to arrive at a sum, wherein the sum is indicative of the presence or absence of an autoimmune disorder in the subject.

17. A method of diagnosing an autoimmune disorder in a subject, the method comprising:
(a) providing an array comprising a plurality of nucleic acid sequences, wherein each nucleic acid sequence corresponds to a known gene;
(b) providing a biological sample derived from the subject, wherein the biological sample comprises a nucleic acid;
(c) hybridizing the biological sample to the array;
(d) detecting all nucleic acids on the array to which the biological sample hybridizes;
(e) determining a relative expression level for each nucleic acid detected;
(f) creating a profile of the relative expression levels for the detected nucleic acids; and
(g) comparing the profile created with a standard profile, wherein the comparing diagnoses an autoimmune disease in a subject.

18. The method of claim 17, wherein the autoimmune disorder is selected from the group consisting of rheumatoid arthritis (RA), systemic lupus erythematosus (SLE), multiple sclerosis (MS), type 1 (insulin-dependent) diabetes (IDDM), and combinations thereof.

19. The method of claim 17, wherein the array is selected from the group consisting of a microarray chip and a membrane-based filter array.

20. The method of claim 19, wherein the array comprises at least two genes represented by SEQ ID NOs: 1-70.

21. The method of claim 19, wherein the array comprises at least five genes represented by SEQ ID NOs: 1-70.

22. The method of claim 19, wherein the array comprises at least ten genes represented by SEQ ID NOs: 1-70.

23. The method of claim 19, wherein the array comprises at least twenty genes represented by SEQ ID NOs: 1-70.

24. The method of claim 19, wherein the array comprises at least twenty-five genes represented by SEQ ID NOs: 1-70.

25. The method of claim 19, wherein the array comprises all of the genes represented by SEQ ID NOs: 1-70.

26. The method of claim 19, wherein the array further comprises at least one internal control gene.

27. The method of claim 17, wherein the biological sample is a cell.

28. The method of claim 27, wherein the cell is a peripheral blood mononuclear cell.

29. The method of claim 17, wherein the subject is an animal.

30. The method of claim 29, wherein the animal is a mammal.

31. The method of claim 30, wherein the mammal is a human.

32. The method of claim 17, wherein the determining comprises a technique selected from the group consisting of a Northern blot, hybridization to a nucleic acid microarray, and a reverse transcription-polymerase chain reaction (RT-PCR).

33. The method of claim 32, wherein the RT-PCR is quantitative RT-PCR.

34. The method of claim 17, wherein the determining is of the expression levels of at least two genes represented by SEQ ID NOs: 1-70.

35. The method of claim 34, wherein the determining is of the expression levels of at least five genes represented by SEQ ID NOs: 1-70.

36. The method of claim 34, wherein the determining is of the expression levels of at least ten genes represented by SEQ ID NOs: 1-70.

37. The method of claim 34, wherein the determining is of the expression levels of at least twenty genes represented by SEQ ID NOs: 1-70.

38. The method of claim 26, wherein the determining is of the expression levels of at least twenty-five genes represented by SEQ ID NOs: 1-70.

39. The method of claim 34, wherein the determining is of the expression levels of all of the genes represented by SEQ ID NOs: 1-70.

40. The method of claim 17, wherein the comparing comprises:
(a) establishing an average expression level for each gene in a population, wherein the population comprises statistically significant numbers of normal subjects and subjects that have one or more different autoimmune disorders;
(b) assigning a first value to each gene for which the expression level in the subject is higher than the average expression level in the population and a second value to each gene for which the expression level in the subject is lower than the average expression level in the population; and
(c) adding the values assigned in step (b) to arrive at a sum, wherein the sum is indicative of the presence or absence of an autoimmune disorder in the subject.

41. A kit comprising a plurality of oligonucleotide primers and instructions for employing the plurality of oligonucleotide primers to determine the expression level of at least one of the genes represented by SEQ ID NOs: 1-70.

42. The kit of claim 41, comprising oligonucleotide primers to determine the expression level of at least five of the genes represented by SEQ ID NOs: 1-70.

43. The kit of claim 41, comprising oligonucleotide primers to determine the expression level of at least ten of the genes represented by SEQ ID NOs: 1-70.

44. The kit of claim 41, comprising oligonucleotide primers to determine the expression level of at least twenty of the genes represented by SEQ ID NOs: 1-70.

45. The kit of claim 41, comprising oligonucleotide primers to determine the expression level of at least thirty of the genes represented by SEQ ID NOs: 1-70.

46. The kit of claim 41, comprising oligonucleotide primers to determine the expression level of at all of the genes represented by SEQ ID NOs: 1-70.

47. The kit of claim 41, further comprising oligonucleotide primers to determine the expression level of a control gene.
